(19) United States
(12) Patent Application Publication
(10) Pub. No.: US 2005/0175505 A1
(43) Pub. Date: Aug. 11, 2005

(54) Personal monitor to detect exposure to toxic agents

(76) Inventors: Hal C Cantor, Farmington Hills, MI (US); Robert W. Hower, Farmington Hills, MI (US); Lucian Cosmin, Farmington Hills, MI (US)

Correspondence Address: Kenneth I Kohn;Kohn & Associates, Suite 410, 30500 Northwestern Highway, Farmington Hills, MI, 48334, US

(21) Appl. No.: 10/508,228

(22) PCT Filed: Mar. 20, 2003
(86) PCT No.: PCT/US03/08575

Related U.S. Application Data
(60) Provisional application No. 60/365,869, filed on Mar. 20, 2002.

(51) Int. Cl. G01N 33/00

(52) U.S. Cl. 422/68.1; 422/100; 422/57

(57) ABSTRACT

A micro-device for testing for agents in a fluid including at least one micro-chamber and a sensor for sensing agents in the fluid, the sensor is located within the micro-chamber. A micro-device for testing for agents in a fluid including a micro-chamber and a micro-fluidic system, the micro-fluidic system is used for pumping the fluid into the micro-chamber. A micro-device for testing for agents in a fluid including a miniature micro-chamber for testing for agents in a small amount of fluid.

### BACKGROUND OF THE INVENTION

1. Technical Field

The present invention relates to the field of micro-sensor technology. More specifically, the present invention relates to a device for detecting and monitoring presence and exposure to environmental agents.

2. Background Art

Humans and animals encounter various environmental agents. Although environmental agents can be harmless, there are numerous agents that are toxic and cause an immunologic response. Thus, it is important to not only identify the presence of these agents, but also to determine whether or not a person or animal has been exposed to the agents in order to provide a more thorough treatment regiment.

Exposure to toxic agents typically occurs in the workplace. Disease from exposure to toxic agents in the work environment causes an estimated 50,000 to 70,000 deaths and 350,000 new cases of illnesses each year in the United States alone. Some of these toxic agents include, but are not limited to: PCBs, lead, neurotoxins, viruses, bacteria, pathogens, and chemicals. So, workers in such environments must be monitored and evaluated as to exposure to and biological load of these agents. Presently, epidemiologists evaluate exposure to various agents by determining the proximity of the worker to a source of the toxin or by large outbreaks of symptoms. Occasionally, side effects of exposure to toxic agents strike and do not manifest until an extended time after exposure. Thus, to monitor acute exposure in a specific population, it is necessary to determine background levels in the general population. Additionally, while the immune systems of most individuals can protect against low levels of exposure to certain toxins or agents, some individuals have weak immune systems and can be more susceptible to deleterious effects from extremely low levels of toxin exposure similar to how some individuals have allergic reactions to antigens while others do not. Therefore, once symptoms resulting from exposure to toxic agents become evident, it is often too late to effect adequate treatment.

Monitoring the presence of toxic agents in the environment or biological load within a human or animal can be done with numerous methods and through various devices known to those of skill in the art. The presence of the agents can be detected directly by merely detecting their presence in interstitial fluid obtained transdermally through the skin surface of a subject or through other fluids obtained from the subject such as urine, perspiration, saliva, tears, etc. Alternatively, monitoring and/or evaluating the subject's immunologic response to the agents can indirectly determine the presence of the agents. Thus, immunoglobin production and a build up thereof can be evaluated.

Additionally, it has been demonstrated that certain toxic agents, such as pesticides, drugs, and many industrial compounds and chemical warfare agents, inhibit cholinesterase (ChE) activity in human blood. It has been demonstrated that depression of ChE activity provides an indication of exposure to a wide variety of toxic nerve agents. These depression effects often persist for up to 100 days after exposure to organophosporous nerve agents. Therefore, monitoring ChE levels provides for a manner in which to monitor exposure of a person or animal to various toxic agents.

Currently, there are numerous personal monitoring systems employed in the workplace to detect airborne particles. Most of these systems are not automated and merely sample the environment near the head of the individual. While this analysis provides an indication of potential exposure of an individual to environmental workplace toxins, it does not actually measure biological exposure or load. Further, these systems require off-line analysis utilizing complex and expensive analysis systems. Moreover, these air monitors are not specifically designed to detect exposure to agents absorbed through the skin, eyes, or methods of entry into the body other than through respiration.

Currently existing monitors also are not designed to monitor long term exposure to low levels of agents. Long-term exposure is a manner in which much of the population obtains a biological load of toxins within tissues, as is typical with polybrominated biphenyl compounds (PBBs) and polychlorinated biphenyl compounds (PCBs).

Other problems associated with currently existing personal monitoring systems include, but are not limited to, requirement of large amounts of samples, use of expensive devices, use of off-line analytical machinery and computers, utilization of large non-miniaturized monitoring systems, and restricted use by those trained to read and operate those systems. Other assay systems utilize toxic compounds, especially radiation as in radioimmunoassays (RIAs).

There are commercially available assay systems that are typically specific to antibody and antigen based assays. These devices include RIAs and Enzyme linked immunosorbent assays (ELISAs). Although specific and sensitive, these assay systems and related methods have numerous drawbacks. For instance, RIAs utilize toxic compounds, specifically radioactive materials and radiation, to label molecules. As a result, large quantities of radioactive waste are produced and expensive equipment, which must be utilized by trained personnel, is required. Further, typical RIAs or ELISAs can take as long as seven days for incubation periods, require trained personnel, as well as expensive, large, and non-transportable detection equipment. As for these previous assays, they require relatively large volumes of sampling fluid, typically a minimum of 100 μL, and large quantities of antibodies and reagents. Further, these assays require separation, purification, and washing steps prior to assaying.

There is therefore a need for an automated, miniaturized device to monitor personal exposure to environmental agents and to identify the body's reactions to these agents. Additionally, there is a need for a device capable of providing a differential blood analysis, for example, of cholinesterase activity or toxin contamination. Further, there is a need for a device that provides sensitivity based on the size of the system and not necessarily based on the sensitivity of the assay.

### SUMMARY OF THE INVENTION

In accordance with the present invention, there is provided a micro-device for testing for agents in a fluid including a micro-chamber and a sensor for sensing agents in the fluid, the sensor is located within the micro-chamber. Also provided is a micro-device for testing for agents in a fluid including a micro-chamber and a micro-fluidic system, the micro-fluidic system is used for pumping the fluid into the micro-chamber. A micro-device for testing for agents in a fluid including a miniature sampling chamber for testing for agents in a small amount of fluid is provided.

### DESCRIPTION OF DRAWINGS

Other advantages of the present invention can be readily appreciated as the same becomes better understood by reference to the following detailed description when considered in connection with the accompanying drawings wherein:

FIG. 1A illustrates an embodiment of the present invention of a one-time use device, wherein the device includes a collection chamber and several assaying chambers, and 1B illustrates another embodiment of the present invention of a system, wherein the system includes at least one sensor connected to a remote display system and at least one collection chamber, at least one separation chamber, and at least one sensing chamber in communication with the other chambers through micro-conduits;

FIGS. 2A and B show the CAD layout of the chambers wherein two chips constitute the top and bottom of the device;

FIG. 3 shows the complete mask layout;

FIG. 4 shows the cross-section of the assembled chip;

FIG. 5 shows top and bottom pieces of the chamber, mated together;

FIG. 6 shows a thick bead of photoresist material at the corner of the etched;

FIG. 7 shows that the vaporized OP was bubbled through an appropriate buffer solution, causing the OP to dissolve back into the liquid to be assayed;

FIG. 8 is a graph that shows the activity of the enzyme was determined by measuring the change in absorbance (or slope) after one month and two months of storage at −4 C;

FIG. 9 shows that the separation of the enzyme globule from the plastic substrate caused the effective surface area of the immobilized enzyme to increase, enabling more substrate to react with the enzyme;

FIG. 10 shows that there was significant suppression of enzyme activity in the 2 μl immobilized enzyme wells;

FIG. 11 shows the results of a kinetic protocol was created on the photometric micro-titer plate reader to take an absorbance reading at 405 nm every minute for 10 minutes, and compute an average slope;

FIG. 12, show almost identical slopes for control and plasma cholinesterase, confirming the capacity of the BTC substrate to detect cholinesterase activity in plasma;

FIG. 13 shows that acetylcholinesterase from RBC lysate had significant activity (slope=53.6 mOD/min) when the AcTC substrate was used, whereas there was significantly less activity (slope=13.7 mOD/min) for the reaction using the BTC substrate;

FIG. 14 shows the effect of selective inhibition on plasma samples that were treated with quinidine (20 μM), the inhibitory effect was observed only when BTC was used;

FIG. 15 shows the effect of selective inhibition on plasma samples that were treated with quinidine (20 μM), the inhibitory effects of cholinesterase activity with and without quinidine was observed;

FIG. 16 shows that diluted and undiluted plasma showed cholinesterase activity using substrate reagents that were dried and spotted individually

FIG. 17 shows that the present invention can include a detection chamber that can fit into a conventional 96 well plate and read using a conventional spectrophotometer;

FIG. 18 shows that absorbance increased in a linear manner for the wells containing plasma and also shows that a detectable color change occurred;

FIG. 19 shows the reliability of the sampling and immunoassay analysis and a correlation to literature values, the pre melatonin saliva values were averaged (n=5, mean=17.5+/−8.4 pg/ml);

FIG. 20 shows that in normal adults, serum melatonin concentrations are highest during the night (about 60 to 200 pg/mL) and lowest during the day (about 10 to 20 pg/mL) and that these concentrations are well within the melatonin standard curve as determined by amperometry;

FIG. 21 shows a glucose (Sigma, Cat. No. EC No 200-075-1, Lot No. 41 K0184) standard curve that was prepared with concentrations ranging from 50 mg/dL to 400 mg/dL;

FIG. 22 shows that the diode acts as a quarter wave stack, enhancing the signal at certain wavelengths;

FIG. 23 shows that the response of the diodes is linear to the amount of incident power;

FIG. 24 shows optical chemical sensors reproduced on silicon chips by incorporating a photo-diode with an optical membrane on top of the diode;

FIG. 25 is a photomicrograph of the 2 μm sensor array;

FIG. 26 shows a different size sensor array chips bonded in a ceramic carrier;

FIG. 27 shows a schematic of the sensor array;

FIG. 28 shows alternative sensor array configurations;

FIG. 29 shows an inhibition of the ChE activity that was demonstrated in the presence of OP;

FIGS. 30A, 30B, 30C, and 30D illustrate a variety of different support mechanisms located within a chamber of the present invention;

FIGS. 31A, 31B, and 31C illustrate a variety of support mechanism spacing within a chamber of the present invention;

FIGS. 32A and 32B illustrate a CAD drawing of a transdermal sampling chamber of the present invention;

FIG. 33 illustrates a micro-fluidic system of the present invention; and

FIG. 34 illustrates a micro-fluidic actuator and micro-fluidic valve of the micro-fluidic system of the present invention.

### DESCRIPTION OF THE INVENTION

Generally, the present invention provides a completely automated, miniaturized device capable of detecting 10 and monitoring different types of agents from a minute amount of fluid. The present invention can determine a subject's reaction to various agents, analyze trends, perform comparisons among a normalized standard of people, determine tolerance levels of a subject, and/or notify or alarm an individual of exposure. More specifically, the present invention is a micro-electro-mechanical system (MEMS) based device 10 with optionally integrated fluid acquisition or micro-fluidic system 11 and external monitoring system 44.

The terms “chamber 12,” “sampling chamber 12,” “reacting chamber 12,” and “sensor chamber 12” are defined as an enclosed cavity wherein fluids are retained.

The term “agent” is defined as a traceable biological or chemical component. As used herein, an “agent” is meant to include, but is not limited to environmental agents, blood markers, antigens, pesticides, drugs, chemicals, toxins, PCBs, PBBs, lead, neurotoxins, blood electrolytes, metabolites, analytes, Na⁺, K⁺, Ca⁺, urea nitrogen, creatinine, biochemical blood markers and components, ChE, AChE, BuChe, tumor markers, PSA, PAP, CA 125, CEA, AFP, HCG, CA 19-9, CA 15x-3, CA 27-29, NSE, hydroxybutyrate, acetoacetate, and any other similar agents known to those of skill in the art.

The term “testing” is defined as detecting, sensing, and/or analyzing an agent. Testing can either determine the presence of the agent or identify the agent itself. Moreover, testing includes both quantification and qualification of the agent.

The term “antigen” or “immunogen” is defined as any substance that is capable of inducing the formation of antibodies and reacting specifically in some detectable manner with the antibodies so induced. Not all antigens however, are immunogens. Examples of an “antigen” include, but are not limited to, immunogens such as viruses, bacteria, microbes, pathogens, HIV, hepatitis, anthrax, cholera, Q-fever, smallpox, tuberculosis, and any other similar biological agents or pathogens known to those of skill in the art.

The term “subject” or “subjects” as used herein is defined as, but is not limited to, humans and animals.

The term “fluid” or “fluids” as used herein is meant to include, but is not limited to, blood, plasma, saliva, urine, sputum, feces, interstitial fluids, tears, sweat, water, and any other similar bodily fluids or other fluids known to those of skill in the art.

The term “label” as used herein is defined as a device that enables the quantitation and quantification of an agent. Examples of labels that can be used in connection with the present invention include, but are not limited to, chemiluminescent labels, luminescent labels, fluorescent labels, calorimetric labels, including, but not limited to, absorption, bioluminescence, and fluorescence, radiolabels, and enzyme labels.

The term “working electrode 16” as used herein is defined as, but is not limited to, an electrode that supplies the potential source for affecting oxidation and/or reduction.

The term “counter electrode 18” is defined as an electrode paired with a working electrode 16, through which an electrochemical current passes equal in magnitude and opposite in sign to the current passed through the working electrode. In the context of the invention, the term “counter electrode 18” is meant to include counter electrodes 18 that can have the dual function as a potentiometric reference electrode (i.e. a counter/potentiometric electrode). The counter electrode 18 is an electrode at which an analyte is electrooxidized or electroreduced with or without the agency of a redox mediator.

The term “amperometric electrochemical sensor” is defined as a device configured to detect the presence and/or measure the concentration of an analyte via electrochemical oxidation and reduction reactions on the sensor. These reactions are transduced to an electrical signal that can be correlated to an amount or concentration of analyte.

The term “electrolysis” is defined as the electrooxidation or electroreduction of a compound either directly at an electrode or via one or more electron transfer agents. An example of this includes, but is not limited to, using Glucose Oxidase to catalyze Glucose oxidation creating oxidized Glucose and Peroxide, where the Peroxide is being measured.

The term “facing electrodes” is defined as a configuration of the working and counter electrodes 16 and 18 in which the working surface of the working electrode 16 is disposed in approximate apposition to a surface of the counter electrode 18.

The term “measurement zone 28” is defined as a region of the sample chamber sized to contain only that portion of the sample that is to be interrogated during an analyte assay.

The term “non-leachable compound” or “non-releasable compound” is a compound, which does not substantially diffuse away from the working surface of the working and/or counter electrodes for the duration of an analyte assay.

The term “redox mediator” is defined as an electron transfer agent for carrying electrons between the analyte and the working electrode, either directly or via a second electron transfer agent.

The term “reference electrode 24” is defined as an electrode used to monitor and account for voltage drop due to medium resistance in amperometric sensors, and supplies a reference potential for comparison in potentiometric electrodes.

The term “second electron transfer agent” is defined as a molecule that carries electrons between the redox mediator and the analyte (See example above).

The term “sorbent material” is defined as a material that wicks, retains, or is wetted by a fluid sample in its void volume and does not substantially prevent diffusion of the analyte to the electrode.

The term “working surface 26” is defined as that portion of the working electrode, which is coated with redox mediator and configured for exposure to sample.

The term “actuator 30” as used herein is defined as, but is not limited to, a device that causes something to occur. The actuator 30 activates the operation of a valve, pump, villi, fan, blade, or other microscopic device. Typically, the actuator of the present invention affects fluid flow rates within a chamber.

The term “closed cavity 52” as used herein is defined as, but is not limited to, a sealed cavity that contains a liquid or solid expanding mechanism 32 that is expanded or vaporized to generate expansion or actuation of a flexible mechanism 34. The closed cavity must be completely sealed in order to contain the expansion therein, and must be flexible on at least one side.

The term “expanding mechanism 32” as used herein is defined as, but is not limited to, a fluid capable of being vaporized and condensed within the closed cavity enclosed by the flexible mechanism 34. The expanding mechanism 32 operates upon being actuated or heated. The expanding mechanism 32 includes, but is not limited to, water, wax, hydrogel (solid or non-solid), hydrocarbon, and any other similar substance known to those of skill in the art. Condensation of the expanding mechanism 32 occurs when the heat, which is generated to induce expansion of the expanding mechanism, is removed by a surrounding medium such as a gas, liquid or solid. Then, once condensation occurs, contraction of the flexible mechanism 34 takes place.

The term “flexible mechanism 34” as used herein is defined as, but is not limited to, anything that is capable of expanding and contracting with the vaporization and condensation of the expanding mechanism. The flexible mechanism 34 must be able to stretch without breaking when the expanding mechanism 32 is vaporized. The flexible mechanism 34 is made of any material including, but not limited to, silicone rubber, rubber, polyurethane, PVC, polymers, combinations thereof, and any other similar flexible mechanism 34 known to those skilled in the art.

The term “heating mechanism 36” as used herein is defined as, but is not limited to, a heating device that is incorporated with the actuator 30 of the present invention. The heating mechanism 36 generates heat to induce expansion of the expanding mechanism. The heating mechanism 36 is disposed adjacently to the flexible mechanism 34 in order to turn on and off and maintaining on and off selective expansion of the expanding mechanism 32. The heating mechanism 36 can be powered using any power source known to those of skill in the art. In the preferred embodiment, the heating mechanism 36 is powered by a battery. However, both AC and DC mechanisms are used to minimize power requirements. Generally, the heating mechanism 36 is formed of materials including, but not limited to, polysilicon, elemental metal, silicide, or any other similar heating elements known to those of skill of the art. Moreover, the heating mechanism 36 is disposed within a medium such as SiO₂ or other solid medium known to those of skill in the art.

The term “temperature sensor 38” as used herein is defined as, but is not limited to, a device designed to determine temperature. A resistive temperature sensor 38 is made from material including, but is not limited to, polysilicon, elemental metal, silicide, and any other similar material known to those of skill in the art. Thermocouple temperature sensor 38s can also be used. Typically, the temperature sensor 38 is situated within or near the heating element of the heating mechanism 36.

The terms “micro-conduit” and “conduit 40” as used herein are defined as, but not limited to, any type of tube, pipe, planar channel, conduit, or any other similar conduit known to those of skill in the art. The conduit has a wall mechanism made from material including, but not limited to, silicon, glass, rubber, silicone, plastics, polymers, metal, and any other similar material known to those of skill in the art. In one embodiment of the micro-fluidic valve, the conduit encompassing the micro-actuator is etched out of glass in a nearly hemispherical shape. A variety of conformations of spherically cut patterns (i.e. ⅓ of a sphere, ½ of a sphere, etc.) with differing radii and footprints are employed to provide different valving characteristics.

The device of the present invention can be composed of numerous materials including, but not limited to, plastic, silicone, glass, metals, alloys, rubber, combinations thereof, or any other similar material known to those of skill in the art. Typically, the device of the present invention is manufactured by chemical etching methods known to those of skill in the art. Thus, the chambers and micro-conduits of the present invention can be etched into a base material of silicon or glass. The chambers are made out of material that is sandwiched between pieces of silicon, glass or membranes. Further, the present invention can be made by utilizing glues and other securing methods and materials known to those of skill in the art. Alternatively, the chambers and conduits can be produced from plastic by injection molding, micro-milling, or soft lithography. The materials of the present invention can be modified or altered according to the specific design required. Moreover, the device of the present invention can vary in size, shape, and configuration without departing from the spirit of the present invention.

The device 10 of the present invention has numerous advantages over currently existing devices. For instance, the present invention is minimally invasive and measures nanoliter and microliter amounts of fluids and not milliliter amounts. The device of the present invention can perform various assays such as ELISA and RIA, but also is capable of performing chromatographic separations. The device 10 of the present invention is capable of performing various tests on a single, small unit sensor system without the aid, or need, of external equipment (i.e., laboratory-on-a-chip). However, the device can be optionally linked to an external electrical source, power source, computer unit, or palm pilot as desired by the user either directly with wires or via telemetry. The device 10 of the present invention can also be constructed as an instrumentless device and can provide easily readable visual indicia of a positive and/or negative test.

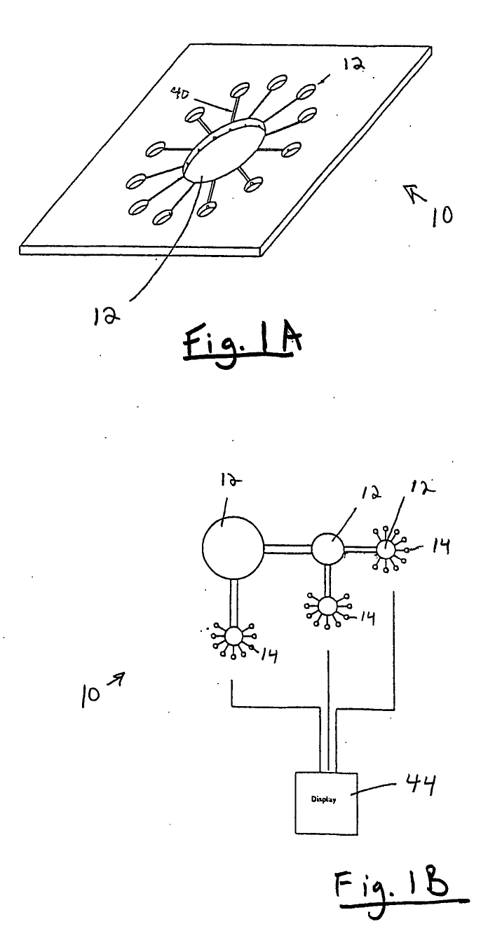

The present invention has additional advantages in that it is capable of having either a single or numerous chambers 12 (FIGS. 1 and 2). Various reactions of the fluid can take place in one chamber 12 or various other chambers 12. Movement of the fluids occurs through micro-conduits 40 connecting the chambers 12. Alternatively, reactions can take place between chambers 12 and within the micro-conduits 40 themselves. For example, a fluid can be added to a sampling chamber 12, treatment of the fluid then occurs along the micro-conduit, and the results are obtained at an end of micro-conduit 40 or the destination site of the fluid. Various treatments of the fluid can take place within the micro-conduit 40 such as degassing, surfactant treatment, heating, incubating, mixing with reagents, and the like that can change the state of the fluid. Additionally, various membrane-based, enzymatic, potentiometry, amperometric, electrochemical, and immunological tests can be performed within the chambers 12 or micro-conduits 40.

The device 10 of the present invention does not require separation and/or purification of fluids before performing assaying as in typical RIA and ELISA assays. All purification and preparation steps can occur within the device of the present invention (e.g., chromatography, primary incubation with antibody, enzymatic degradation, blood cell separation, blood cell lysis, and the like). Additionally, the device 10 of the present invention is smaller than any other system that is utilized to perform conventional RIA or ELISA based assays. The present invention utilizes and requires significantly fewer quantities of antibodies, reagents, chromophores, samples, physical space, energy, and incubation time. The microscopic nature of the device of the present invention is more amenable to temperature regulation; thus, making the assays more precise and accurate, as well as reducing incubation periods (e.g., temperature control can be performed on the device to utilize integrated polysilicon heaters and thermocouples/thermistors). The size of the device 10 also allows multiple assays to be run on a single dipstick-type device to provide color-coded testing results more useful for the layperson via in-home testing. Thus, multiple background, standards, sample duplicates, and the like can all be performed on a 1×1 inch device, which increases accuracy through statistical analysis. Alternatively, the device can be of a smaller size such as in the micro or nano range.

As mentioned above, the device 10 of the present invention utilizes significantly less power than conventional micro-fluidic devices. It is compatible with standard CMOS fabrication and therefore the controlling circuitry can be integrated onto the substrate. It is calculated that less than 700 μW of power is necessary to achieve a pumping rate of 10 μL/min and that pumping rates of 100 μL/min are achievable with this design. Pumping volumes are accurate to within 5 nL volumes.

The device 10 of the present invention has numerous embodiments. One embodiment is directed towards a micro-electro-mechanical system (MEMS) based device 10 including at least one sampling chamber 12. The device can optionally include micro-conduits 40, sensor arrays 14, a micro-fluidic system 11, and an external monitoring system 44. The device 10 can simply include one or multiple chambers 12 (i.e., sampling, reacting, and/or sensing). If there are multiple chambers 12, then they can be in communication with each other via micro-conduits 40. Alternatively, other embodiments are directed towards a device 10 including a sampling chamber connected to either reaction chambers 12 and/or sensor chambers 12 having sensor arrays 14. In any of the embodiments of the present invention, the system or device 10 can be placed on an attachable means such as a patch, Band-Aid, or other disposable sensor system. The device 10 can be placed directly onto the skin of a subject in order to obtain samples.

The chamber 12 (i.e., sampling, reacting, and/or sensing) of the present invention is generally illustrated in FIGS. 1 and 2. The chamber 12 provides for an area for placing the fluid, performing chemical reactions, sensing or detecting agents within the fluid, and/or collecting or storing the fluid. A simple one-step process can occur in one or more of the chambers 12. If numerous chambers 12 are utilized, these chambers 12 can perform required separations, measurements, and analyses of the fluid. For example, the chamber 12 can be used to lyse whole cells such as red blood cells by utilizing salts, chaotropes, heat, and any other similar reagents known to those of skill in the art. Additionally, certain chambers 12 can be utilized to contain just cells, while other chambers 12 contain only plasma therein. The actual structural components of the chambers 12 are outlined below and illustrated in the attached figures.

The chamber 12 can have various designs that have a flap or membrane covering the chamber 12 therein as well as configurations of supports 46 to act as stand-offs to prevent occlusion by the skin or to increase mixing and disrupt flow of the fluids therein. The supports 46 can vary in size and shape. For example, the bottom of the supports 46 can have a teardrop shape, oval shape, triangular shape, square, rectangular, cylindrical, and the like, while the top of the supports 46 is narrower or the same size and shape as the bottom portion thereof. The supports 46 also vary in size (i.e., volume) and shape in order to increase the volume capacity of the chamber 12.

The fluids within the device 10 of the present invention primarily move via mechanisms including, but not limited to, capillary action, diffusion, micro-fluidic pumps, gravity, mechanical action, peristaltic action, pneumatic action, and any other similar mechanism known to those of skill in the art. The fluids can initially diffuse through membranes located on the device of the present invention and into various chambers 12. In other embodiments, there is no movement through a membrane. The fluids move from chamber 12 to chamber 12 and within micro-conduits 40. Alternatively, active mechanical pressure induced by micro-fluidic pumps 46 can aid in the movement of the fluids. For instance, positive or negative pressure on a membrane flap can move the fluids or active mechanical movement of micro-pumps 46 or actuators 30 can provide enough force to drive the fluids.

The microconduits 40 can be made of numerous materials as listed above. Additionally, the microconduits 40 can contain within the liner of the tube, placed in the tube or within the tube materials itself, various chemicals or reagents. The chemicals or reagents that are contained within the micro-conduits 40 or impregnated within the micro-conduits 40 itself vary according to desired outcomes and reactions. For instance, the micro-conduits 40 can be coated with heparin to prevent clotting of blood, any surfactant to prevent bubbling of the fluid sample, charcoal to separate steroids, and any other similar substances known to those of skill in the art. Moreover, the micro-conduits 40 can be used to perform various treatments or reactions so that as the fluid sample travels along the micro-conduits 40, the reaction or treatment occurs and thus by the time the fluid sample reaches a designated chamber 12 or other location, the reaction or treatment is finished.

As discussed above, the device 10 of the present invention can also include a micro-fluidic system 11 that aides in the quantitative and/or qualitative determination of the fluid samples. The microfluidic system 11 includes various components including, but not limited to, microfluidic pumps 46, micro-fluidic devices 48, additional chambers 12, micro-fluidic valves 50, micro-fluidic actuators 30, DNA chips, ports, miniature conduits or tubes 40, electrodes, and deflectable membranes made of materials such as glass, plastic, rubber, and any other similar materials known to those of skill in the art. A more detailed description of the micro-fluidic system is set forth in PCT/US01/27340, filed Aug. 31, 2001, which is incorporated herein by reference.

The micro-fluidic system 11 includes actuators 30, which are the driving mechanism behind various components of the micro-fluidic system 11. The micro-fluidic valves 50 have various pressures and temperatures required for their actuation. The peristaltic pump 46 is selectively controlled and actuated through an integrated CMOS circuit or computer control, which controls actuation timing, electrical current, and heat generation/dissipation requirements for actuation. Integration of control circuitry is important for the reduced power requirements of the present invention. Closed loop feedback provides the basis of automated adjustment of circuitry within the micro-actuator 30.

The actuator 30 includes a closed cavity 52, flexible mechanism 34, and expanding mechanism 32. Fabrication of actuators 30 is accomplished by generating electron-beam and/or optical masks from CAD designs of the micro-fluidic system. Then, using solid-state mass production techniques, silicon wafers are fabricated and the flexible mechanisms 34 for the actuators 30 are subsequently placed on the chips.

In the micro-fluidic system 11 without integrated circuitry, the control circuitry is produced on external breadboards and/or printed circuit boards. In this manner, the circuitry is easily, quickly, and inexpensively optimized prior to miniaturization and incorporation as CMOS circuitry on-chip that can be controlled manually, or through the use of a computer with digital and analog output. Optimized CMOS circuitry, modeled utilizing CAD solid-state MEMS and CMOS design and simulation tools, is integrated into the active device making it a stand-alone functional unit.

Using an arbitrary waveform generator, and/or computer controlled digital-to-analog (d/a) and analog-to-digital (a/d) PCI computer cards (for example, the PCIMIO16XH, National Instruments) the optimal operating parameters (i.e., stimulatory waveform patterns) are configured to generate peristaltic pumping action. Electronic control of the actuators 30 is optimized to maximize flow rates, maximize pressure head, and minimize power utilization and heat generation. Another parameter that is evaluated includes the temperature profile of the medium being pumped. To minimize power consumption and heat generation, a resistor-capacitor circuit is utilized to exponentially decrease the voltage of the sustained pulse. Further, integrated circuitry initiation and clocking of the circuitry provide control of the second-generation actuators.

An e-prom is also included on-chip to provide digital compensation of resistors and capacitors to compensate for process variations and, therefore, improve the process yield. Electrical access/test pads are designed into the chips to allow for the testing of internal nodes of the circuits.

The flexible mechanism 34 deflects upon the application of pressure thereto. In one embodiment, the flexible mechanism 34 is screen-printed over the expanding mechanism 32 utilizing an automated screen-printing device, a New Long LS-15TV screen-printing system. The flexible mechanism 34 is very elastic and expands many times its initial volume as the expanding mechanism 32 under the flexible mechanism 34 is vaporized. Due to the large deflection, it is possible to completely occlude a micro-conduit 40 with this flexible mechanism 34, hence providing the functionality of an electrically actuated microscopic valve 50. The present invention can also apply the flexible mechanism 34 with syringe or pipette devices or spin coat it on the entire wafer. Photo curable membrane can also be used to pattern the flexible mechanism 34 on the wafer.

A wide variety of commercially available polymers can be utilized as the flexible mechanism 34, including, but not limited to: Polyurethane, PVC, and silicone rubber. The actuator flexible mechanism 34 must possess elastomeric properties, and must adhere well to the silicon or other substrate surface. A material with excellent adhesion to the surface, as well as appropriate physical properties, is silicone rubber.

In an embodiment of the micro-fluidic system 11, the flexible mechanism 34 is made of silicone rubber. The silicone rubber can be dispensed utilizing automated dispensing equipment, or can be screen-printed directly upon the silicon wafer. Screen-printing methods have the advantage that the entire wafer, containing hundreds of pump and valve actuators, can be produced at once. By varying the amount of solvent in the polymer, such as silicone rubber, the flexible mechanism 34 thickness and its resulting physical force characteristics can be precisely controlled.

The flexible mechanism 34 can serve the dual purpose of actuation as well as serving as the bonding material used to attach the liquid flow channels to the silicon chip containing the actuators. By covering the entire area of the chip with the flexible mechanism 34, with the exception of the sensing regions and the bonding pads, the glass or plastic channels can be “glued” to the actuator containing silicon chip. This method provides additional anchoring and strength to the actuation flexible mechanism 34, and allows the actuation area to encompass the entire actuation chamber. The only drawback to this method is potential protein and/or steroid adsorption onto the micro-conduits 40. However, with proper flexible mechanism 34 selection and chemical treatment, molecular adsorption can be minimized, or a second, thin, inert layer can be used to coat the flexible mechanism 34.

The expanding mechanism 32 selectively expands the cavity defined by the flexible mechanism 34 thereof and thereby selectively flexes the flexible mechanism 34. The expanding mechanism 32 can be made of various materials. In one embodiment, the expanding mechanism 32 is a hydrogel material, which contains a large amount of water or other hydrocarbon medium, which is vaporized by the underlying heating mechanism 36. In this embodiment, the volume of hydrogel needed to produce the desired actuation and pressure for the flexible mechanism 34 is approximately 33 pL. With this design, approximately 97% of the energy generated by the heating mechanism 36 is transferred into the hydrogel for vaporization.

A practical technique for the micro-fluidic pumping of moderate volumes of liquid is through the use of peristaltic pumping utilizing pneumatic actuation. The integrated micro-fluidic pumping system 11 of the present invention is designed to sample small amounts of interstitial fluid from the body on a continuous basis. In order to analyze the microscopic volumes, silicon micro-machining methods and recent improvements in membrane deposition technologies are utilized to produce a microscopic test chamber 60 on the order of 50 nL in volume, roughly 3-4 orders of magnitude less volume than current systems. In addition to the improved response time, the reduction to microscopic volumes allows the use of very small amounts of calibration solution to effect calibration and rinsing, hence reducing the overall size of the package. In some systems the calibration solutions are a significant portion of the entire package (Malinkrodt Medical/L) where, even though miniature sensors are used, liters of calibration solutions are necessary.

In one embodiment, the micro-fluidic pump 46 design is based upon electrically activated pneumatic actuation of a micro-screen printed silicon rubber membrane. Generally, the pump includes the micro-fluidic actuator 30 including a closed cavity 52, flexible mechanism 34 defining a wall of the closed cavity 52, and expanding mechanism 32 disposed within the closed cavity. The flexible mechanism 34 deflects upon the application of pressure thereto and the expanding mechanism 32 selectively expands the cavity and thus flexible mechanism 34 and thereby selectively flexes the expanding mechanism 32.

The micro-fluidic actuator 30 is based upon electrically activated pneumatic actuation of a micro-screen-printed or casted flexible mechanism 34. The peristaltic pump generally includes three actuators 30 placed in series wherein each actuator 30 creates a pulse once it is activated. By working in tandem, the actuators 30 peristaltically pump fluids. The optimal firing order and timing for each actuator 30 depends upon the requirements for the system 11 and are under digital control to create the peristaltic pumping action.

The advantage of pneumatic actuation is that large deflections can be achieved for the flexible mechanism 34. To actuate the flexible mechanism 34, a vaporizable fluid is heated and converted into vapor to provide the driving force. Utilizing an integrated heating mechanism 36, the expanding mechanism 32 is vaporized under the flexible mechanism 34 to provide the pneumatic actuation. This actuation occurs without the requirement of utilizing external pressurized gas.

The liquid or gaseous fluid being pumped serves the purpose of acting as a heat sink to condense the vapor back to liquid and hence return the flexible mechanism 34 to is relaxed state when the heating mechanism 36 is inactivated. A temperature sensor 38 is integrated adjacent to the actuator to monitor the temperature of the micro-fluidic integrated heating mechanism 36 and hence, expanding mechanism 32.

Once the heating mechanism 36 is activated, vaporization of the expanding mechanism 32 takes place. The expanding mechanism 32 component imposes a pressure upon the flexible mechanism 34 causing it to expand and be displaced above the heating mechanism 36 and reduce the volume of the chamber. This methodology can be utilized to displace fluid between the flexible mechanism 34 and the walls of the chamber (pumping action), to occlude fluid flow through the chamber (valving action), to provide direct contact to the glass substrate to effect heat transfer, or to provide the driving force for locomotion of a physical device (i.e., as in a walking caterpillar and/or a swimming paramecium with a flapping flagella, in which case the glass chamber encompassing the micro-actuator 30 is not used).

In one embodiment, the temperature of the saturated liquid hydrogel, at 1 ATM, is assumed to be 100° C. The heat flux to the air, through the back of the heating mechanism 36, is calculated to be 1263 W/K-m². The total heat flux through the device is calculated to be 46,995 W/K-m² with a total flux from the heating mechanism 36 of 47,218 W/K-m² (i.e. 97% efficiency of focused heat transfer). In this embodiment, the temperature of the inactive state hydrogel varies between 86° C. and 94° C.

The temperature of the activated, vapor state hydrogel is approximately 120° C., which is the saturation temperature for steam at 2 ATM. The heat transfer coefficient for convection can be calculated directly from the thermal conductivity. The heat flux to the air through the back of the heating mechanism 36 is 2818 W/K-m². The heat flux through the device is 21,352 W/K-m² with a total flux from the heating mechanism 36 of 24,170 W/K-m². When the aqueous component of the hydrogel is completely in the vapor state, there is no fluid in the channel and the thin film of solution between the flexible mechanism 34 and the glass is approximately at 60° C. These values and calculations vary according to the type of actuator, valve, pump, and micro device being used.

In an embodiment of the present invention, the volume of the expanding mechanism 32, in this case, liquid hydrogel, is determined based on the volume of vapor needed to expand the flexible mechanism 34 completely at 2 ATM using the ideal gas law. This assumption is valid because the temperatures and pressures are moderate. The volume of liquid hydrogel necessary to achieve this volume of gas at this pressure, assuming the hydrogel is 10% water and all of the water is completely evaporated, is 0.033 nL. Cylindrically shaped sections of hydrogel are utilized within the actuator 30. This shape has been chosen to optimize encapsulation by the actuator flexible mechanism 34. The cylinders have either a diameter of approximately 140 μm and a height of 2.14 μm, or a diameter of 280 μm with a height of 0.54 μm (identical volumes, different orientation to the heating element). Of course, the shapes and volumes vary according to the type of expanding mechanism 32 being used. For example, photocurable liquid hydrogels have different parameters.

The heating mechanism 36 is poly-silicon, but can be any similar material or mechanism, such as direct metals, known to those of skill in the art. Because of its high thermal conductivity, the silicon substrate acts as a heat sink. To reduce thermal conduction to the silicon substrate, a window in the silicon, located beneath the heating mechanism 36, provides the expanding mechanism 32 with an isolated platform. This window is only slightly larger than the heating mechanism 36 to maintain some thermal conduction to the substrate. After the actuator 30 is energized, thermal conduction to the silicon provides decreased time to condense the liquid in the expanding mechanism. This decreases constriction time and provides improved pumping rates. If the window is significantly larger than the actuator 30, there is no heat conduction path to the substrate, hence increasing condensation time and decreasing the maximal flow rate.

Fabrication of the micro-fluidic system 11 components is based upon the development of a process flow. The fabrication process utilizes bulk silicon micro-machining techniques to produce the isolation windows, and thick film screen-printing techniques, spin coating, mass dispensing, or mechanical dispensing of actuation membranes.

A polymeric hydrogel (or hydrocarbon) can be utilized to provide a physically supportive structure that withstands the application of flexible mechanism 34 as well as to provide the aqueous component required for actuation. Several commercially available materials meet these requirements. A hydrogel is selected that contains approximately 30% aqueous component that vaporizes near 100° C. Several materials have been identified, each of which is suitable in this application, including, but not limited to, hydroxyethylmethacrylate (HEMA) and polyvinylpyrrolidone (PVP). Additionally, hydrocarbons can be used since they possess lower boiling points than aqueous hydrogels, and therefore require less power to effect pneumatic actuation.

Dispensing hydrogel (or hydrocarbon) into the desired location is accomplished utilizing one of three methods. First, a promising method for patterning the hydrogel is to utilize a photopatternable-crosslinking hydrogel. The hydrogel is cross-linked by incorporating an UV photo-initiator polymerizing agent within the hydrogel that cross-links when exposed to UV radiation. Using this technique, the hydrogel is evenly spun on the entire wafer using standard semiconductor processing techniques. A photographic mask is then placed over the wafer, followed by exposure to UV light. After the cross-linking reaction is completed, excess (non-cross-linked hydrogel) is washed from the surface.

The second method involves dispensing liquid hydrogel into well rings created around the poly-silicon heating mechanism 36. These wells have the ability to retain a liquid in a highly controlled manner. Two photopatternable polymers have been utilized to create microscopic well-ring structures, SU-8 and a photopatternable polyimide. These well rings can be produced in any height from 2 μm to 50 μm, sufficient to contain the liquid hydrogel. Once the hydrogel solidifies, flexible mechanisms 34 can be deposited over them. This can be accomplished in an automated manner utilizing commercially available dispensing equipment.

In a third alternate method, a pre-solidified hydrogel is used that has been cut into the desire size and shape. This is facilitated by extruding the hydrogel in the desired radius and slicing it with a microtome to the desired height, or by spinning the hydrogel to the desired thickness and cutting it into cylinders of the desired radius. Utilizing micromanipulators, the patterned gel is placed in the desired area. This process can also be automated.

It is assumed that the temperature on both sides of the SiO₂ that encapsulates the heating mechanism 36 is constant, and that heat flux in each direction is dependant upon the heating mechanism 36 temperature and both sides are resistant to heat flow either through the device or to an air pocket on the heating mechanism 36 backside. Steady-state heat flow through the entire actuator, for the fully actuated state, the intermediate state, and the resting state are modeled. These data are calculated for the static case during which time no fluid flow is occurring (i.e. steady-state; the system is poised at 100° C., waiting to be initiated). The fluid temperature is greater for the contracted state since the liquid hydrogel conducts heat at a greater rate than vapor. Once fluid flow is initiated, the temperature of the solution is raised by only a few degrees Celsius.

A typical problem experienced with many micro-fluidic designs revolves around the methodology for mixing of solutions and reagents. The micro-fluidic peristaltic pump 46 design of the present invention provides mixing action in concert with the pumping action. To construct the micro-fluidic valves 50 and pumps 46 in a manner compatible with the sensor technologies and to integrate the entire system on a single silicon chip, the pump is preferably fabricated using planar MEMS technologies that do not require special wafer bonding, although other methods of fabrication can also be used as are known to those of skill in the art.

For encapsulating a liquid within a silicone rubber membrane, micro-machining techniques, including wafer bonding of multiple chips, are used by others to create a cavity where the liquid is stored. This requires several machining steps to produce the actuators reducing the overall yield of functional pumps and valves, and increasing the cost.

By properly placing the planar actuators within the fluidic channels, micro-pumps, fluidic multiplexers, and valves can be formed. CAD/CAM tools are used to design the photo-masks. This can be accomplished in conjunction with the design of the fluidic channels, ports, and test chambers.

The pneumatically actuated membrane is utilized to produce the micro-fluidic valves. The micro-fluidic actuator's silicone rubber membrane is very elastic and expands many times its initial volume as the liquid under the membrane is vaporized. At least two techniques for the valving of solutions can be used.

The first utilizes the flexible mechanism 34 actuation to completely fill a micro-fluidic channel when actuated, hence providing the functionality of an electrically actuated microscopic valve. The second utilizes the flexible mechanism 34 to occlude an orifice to block fluid flow.

The pneumatically actuated membrane is also utilized to produce the micro-fluidic pumps 46. The micro-fluidic actuator's flexible membrane 34 is very elastic and expands many times its initial volume as the liquid under the membrane is vaporized. The microconduits 40 are designed such that all media flow is in the laminar regime while minimizing fluid volume, dead volume, and residence time. Further, the routing of the microconduits 40 are designed such that the required calibration and wash solutions can be routed into the sensing chamber 12. The conduits 40 and sensing chamber 12 accommodate approximately 50 nL volumes of solution.

Once modeled and optimized, photomasks are created for the fluidic system. Valves at the various ports are optimally designed to start and stop the flow of the various calibration and wash solutions.

In one embodiment, the integration of a sampling system or microfluidic system 11 to the device 10 allows transdermal-sampling techniques for the acquisition of interstitial fluids. This sampling chamber 12 has a maximized surface area within the confines of the device 10 and an extremely minute volume to reduce the required sample volume and to decrease the sampling time. This chamber 12 is micro-machined into the backside of the glass fluidic channel chip.

For mobile applications, automated control of the pumps, valves, and sensors is required to continuously monitor and calibrate the microscopic “lab-on-a-chip” devices. Using integrated electronics, the sensors 14 can be calibrated on a regular basis in an automated manor that is transparent to the user, ensuring accuracy of the data obtained. The sensing system also requires integrated circuitry to buffer the signals, reduce noise, transduce the chemical concentrations into electronic signals, and analyze the signals, allowing untrained personnel to utilize the device.

Another application for integrated circuitry is for the telemetric communication of the device with a base unit, which can then relay the information to a remote location. Moreover, the circuitry can perform closed-loop feedback control for biological applications. For example, closed-loop feedback control can be used to inject insulin into an individual when the transdermal sensor system detects hyperglycemic levels of glucose in the transdermally sampled interstitial fluid, thereby maintaining euglycemia.

The sensor arrays 14 are fabricated in a three-mask process with two metal layers, silver and platinum. Since these metals are difficult to etch using wet chemistry, a resist lift-off process was used to pattern them. This provided an additional advantage in allowing the use of layered materials in a metal structure to modify electrode properties and still allowed for patterning to occur in one step.

Additionally, other sensor 14 conformations can be produced in accordance with the present invention, each with differing transduction and membrane encapsulation properties. These designs incorporate rectangular, circular, and concentric circle shaped electrodes.

In any embodiment, the valves 50 of the present invention utilize an actuating mechanism to occlude a micro-conduit 40 and thereby decreasing or preventing fluid flow. The ability to occlude is selective, in that the valve can effectively open and close a passageway of the micro-conduit 40. The micro-fluidic actuators 30 are the driving mechanism behind the micro-fluidic valves 50 of the present invention.

For a mono-stable valve 50, it is assumed that the temperature on both sides of the SiO₂ that encapsulates the heating mechanism 36 is constant, and that heat flux in each direction is dependent upon the heating mechanism 36 temperature and the general resistance to heat flows either through the device or to the air from the backside. In order to isolate the heater, a cavity is etched in the backside of the wafer, providing thermal isolation. The mono-stable valve 50 requires continuous power to maintain a closed-stated position. Utilizing the heating mechanism 36, an expanding mechanism 32 is vaporized under the encapsulating flexible mechanism 34 thereby providing the pneumatic driving force required for expanding the flexible mechanism 34 and hence occluding the micro-conduit 40. The mono-stable, normally open valve 50 utilizes a single actuator to effectively actuate the valve. As the hydrogel is expanded, the silicone rubber of the actuator completely occludes the micro-conduit 40 to effect valving of the solution. While the normally open valve 50 is less complicated to construct, it requires continuous power or pulsed power to keep the valve closed.

A bi-stable valve 50 is also capable of being utilized. The bi-stable valve 50 is designed that utilizes lower power consumption and a wax material to provide passively open and passively closed functionality, i.e. bi-stability. Thus, power is only required to transition from one state to the other. The bi-stable valve design is based upon the utilization of a moderate melting point solid, such as paraffin wax, which possesses a melting point between 50° C. and 70° C.

The bi-stable valve 50 similarly utilizes actuating mechanisms to occlude the micro-conduit 40. The mono-stable valve 50 can only provide the functionality of a normally open valve. During the period that the valve must be maintained in a closed position, continuous power must be applied. The bi-stable valve 50 utilizes micro-fluidic actuators 30 to provide both zero-power open and closed functionality.

The bi-stable valve 50 utilizes a total of three micro-fluidic actuating mechanisms 30. Any number of actuating mechanisms 30 can be used without departing from the spirit of the present invention. Two actuating mechanisms are physically connected by a micro-conduit 40 formed under the membrane and are filled with a low melting point solid such as paraffin wax as opposed to an aqueous hydrogel (see above for mono-stable actuation). The third is a standard design micro-actuator filled with an aqueous hydrogel connected by the expansion chamber to the middle wax filled actuator. The first two micro-actuators 30 are activated causing the wax to melt. The third, standard, micro-actuator is then activated, providing pneumatic force on the wax containing actuators, causing the orifice containing chamber to close. The wax is then allowed to solidify. Again, the advantage of this valve is that it requires power only to transform from the stable open to the stable closed state.

In the open state, medium in the channel readily flows. To switch from the open state to the closed state, the wax is melted and the pneumatic actuator 30 on the right is expanded. This creates pressure outside the middle actuator, forcing the paraffin into the smaller left chamber, expanding the membrane, thereby blocking fluid flow. The wax is allowed to solidify, after which the power can be removed from the actuator providing the driving force pressure, resulting in an electrically passive closed state. To transition from the closed state to the open state, the wax is melted and membrane tension forces the wax from the small left chamber back into the middle chamber. The micro-valve design provides bi-stable functionality, which only requires power to switch between each state, but is completely passive once in either the open or closed position.

The use of polydimethylsiloxane (PDMS) in multiple layers to directly produce the three-dimensional structures of the micro-fluidic system is a technique well suited to mass production. This technique has the advantages of allowing an entire wafer of chips to be packaged simultaneously and of being compatible with integrated circuitry. This process is fairly complex, requiring multiple photo patterning of the devices and the application of a top layer to complete the structure. Despite the manufacturing challenges, this method is capable of creating three-dimensional micro-fluidic systems.

The sensors 14 of the present invention include at least one amperometric sensor, and at least one potentiometric sensor. The sensors of the present invention can detect neuronal action potentials and the resulting release of neurotransmitting and/or hormones. The sensors can also detect the diffusion, dispersion, degradation, and re-uptake of neurotransmitters, hormones and/or other cellular metabolites. Examples of such sensors 14 are known to those of skill in the art and more specifically, sensors are disclosed in co-pending U.S. patent application Ser. No. 10/111,964, filed May 2, 2002.

Coulometry is the determination of charge passed or projected to pass during complete or nearly complete electrolysis of an analyte, either directly on the electrode or through one or more electron transfer agents. The current, and therefore analyte concentration, is determined by measurement of charge passed during partial or nearly complete electrolysis of the analyte or, more often, by multiple measurements during the electrolysis of a decaying current and elapsed time. Once the hydration shell has been established around the electrode, the decaying current results from the decline in the local concentration of the electrolyzed species caused by the electrolysis. A compound is immobilized on a surface 26 when it is physically entrapped on or chemically bound to the surface.

Electrochemical detection, specifically amperometry, has been used in the past in relatively unsophisticated applications, for example detecting and quantifying eluted molecules at the end of chromatographic columns (Kissinger et al, 1984). The main limitations of amperometry are its low specificity and sensitivity. The present invention takes advantage of this technique's speed and overcomes its limited specificity and sensitivity. First, to enable the amperometric sensors 20 to detect multiple neurotransmitters independently, the sensors employ two particular forms of amperometry; cyclic and constant voltage voltammetry. Second, utilizing a micro-screen printing device, such as a New Long LS-15TV, several different selectivity membranes can be applied over the individual sensors to eliminate background measurement of unwanted compounds (such as ascorbic acid) and impart specificity onto the microscopic electrodes including the sensor (Goldberg et al, 1994). Finally, by encapsulating the multi-site sensor array 14 leads with silicon nitride, which is a substrate that neurons can be made to readily attach, the sensor array is in very close apposition to the secreting neurons allowing measurement of the relatively high neurotransmitter concentrations in the immediate vicinity of the axon, prior to degradation, dilution, dispersion, and re-uptake.

An amperometric process, cyclic voltammetry, is a technique whereby a cyclically repeated triangular waveform of potential is applied between the working and counter electrodes. Individual analytes, such as neurotransmitters, have characteristic oxidation and reduction potentials based on their chemical moieties (Adams, 1969; Dryhurst et al, 1982). When the voltage between the electrodes reaches the oxidation potential of a particular neurotransmitter that molecule oxidizes. Oxidation is a process whereby an electron is stripped from the molecule. The counter electrode absorbs the oxidatively produced electrons, effectively transducing chemistry into electricity. The flow of electrons per unit of time is current, which is proportional to the number of molecules being oxidized. The voltage at which this oxidatively produced current is obtained provides information useful for identifying the analyte such as neurotransmitter, hormone or cellular metabolite being measured (Dryhurst et al, 1982; Baizer et al, 1973).

Other embodiments of the sensor array can include, but is not limited to, additional components such as various separating and purifying mechanisms, heating elements to aid in the lysis of cells, adding and mixing mechanisms, and degassing mechanisms to remove air bubbles. Moreover, various agents can be added to the present invention including, but not limited to, surfactants, primary antibodies to start ELISA reactions, other enzymes to start desired reactions, color reporters (HRP), luminescent agents, or other indicators, and any other chemicals or substances known to those of skill in the art.

In another embodiment of the present invention, the device can be used in conjunction with a hand-held reader for electronically timing the reaction rates and provide digital read-out to automate the measurement process so as to eliminate the need for trained personnel. In this embodiment, the device includes a disposable cartridge containing the enzyme chemistry reagents, detection chambers, and microconduits, a reader containing the sensors, actuators and controlling electronics, and a hand-held read-out system.

The hand-held read out system is usable by both the clinician as well as the patient themselves. It can be designed and developed for use with the device of the present invention. The readout device can be designed as a “hand-held” readout and controlling instrument (RCI) utilizing commercially available Palm or Windows CE hand-held computers. The RCI can be utilized to provide an ergonomic display of sensor and calibration data as well as to monitor trends in the patient. The RCI can control the actuator timing to obtain more or less frequent samples and/or calibrations in a given time period. The RCI unit is also responsible for sensor data conversion utilizing the calibration parameters.

On the chip-based sensor unit, the data is stored in a digital manner until it is ready to be read by the RCI. The RCI accepts a stream of data from the sensor unit and display it in one of two different configurations. The first software implementation in the RCI is for the patient that can display subjective data. In other words, if concentrations are in a high, normal, or low range, then trend analysis providing simple exposed/not-exposed information to the patient. The second version can be utilized by the clinician or trained personnel, who can receive a readout that displays quantitative data from the sensor array and allows data output for use in any standard database or graphing program. In addition, the RCI allows the clinician to control the acquisition device, including sampling frequency, calibration frequency, alarm settings, etc. Numerical concentration levels and trends can be displayed on a hand-held computer or PDA. Furthermore, compatible integration into a Medical database for the individual can take place.

The present invention can be used to detect the presence of various agents and substances as described above. Additionally, the present invention can detect and determine whether exposure to an agent has occurred through the detection of antibody presence and levels thereof. Additionally, the present invention can be used to detect the biological effect of exposure to such various agents and substances as described above.

The device of the present invention is capable of directly determining the presence of an agent, the presence of a reaction to an agent, and providing a differential analysis of an agent level. For example, the device is capable of providing a differential blood ChE analysis. Thus, the device provides a full analysis of a patient's cholinesterase levels using a single drop of blood obtained from finger prick sampling. The device is automated such that minimally trained personnel can utilize it, and provides results in approximately 5 minutes or less. Additionally, the device specifically can monitor acetylcholinesterase (AChE) levels within red blood cells (RBCs) and butyrylcholinesterase (BuChE) levels within plasma. The device is capable of performing these tests within a few minutes and with less than a 5 μl sample of capillary blood.

To accurately monitor exposure in individuals, who are potentially exposed to toxic agents, the individual is tested prior to deployment such that each individual serves as his/her own control. However, if the individual is not tested prior to potential exposure, then average base-line levels can be used for comparison. In essence, the device of the present invention provides all of the functionality of the TEST-MATE™ OP KIT, but is completely automated and miniaturized such that it is field deployable, faster to enable screening of a larger number of samples per day, and have a reduced cost per sample.

The device of the present invention can also perform automated blood cell and plasma separation, utilizing such minute volumes of a sample. There are numerous processes that can be used to perform the separation. For instance, semi-permeable membranes can be utilized including, but not limited to, nitrocellulose, cellulose acetate, nucleopore membranes, rubber, and any other similar membranes known to those of skill in the art. Typically, these membranes have a high percentage of porosity, with pores slightly smaller than the red blood cells (RBCs). Then, once separated, the RBCs are chemically lysed for analysis. In this manner, the device has the ability to monitor plasma BuChE and red blood cells AChE independently.

Alternatively, the blood sample can be automatically separated into two separate assay chambers that are still integrated into the single device. In one of the chambers, the cells are chemically lysed while in the second chamber the blood is left whole. Assays for BuChE and AChE can be conducted for both systems by using the appropriate inhibitors and a comparison is made between the lysed and the non-lysed side to calculate AChE from within the RBCs and BuChE from the plasma, uniquely. More accurately, by measuring the RBC cholinesterase, the plasma cholinesterase is inhibited specifically by guinidine or other plasma cholinesterone inhibitors.

Lyophilized enzyme detection chemistries can be incorporated into the device in the form of membranes on the assay pads. The membrane coated assay pads undergo colorimetric changes in response to analyte concentration. The device incorporates various microscopic, solid-state, photo diode sensors that can be plugged into a hand-held or laptop computer to objectively monitor the assay results. Alternatively, potentiometric and/or amperometric sensors can be employed. Thereby, simple assays or complex enzyme or antibody assays can be utilized.

The device of the present invention can be used in a variety of settings including, but not limited to, health clinics, emergency rooms, hospitals, clinical settings, home health care market, offices, work places, points of chemical exposure including possible terrorist attack sites such as in planes, trains, buildings, and any other similar settings requiring the monitoring or screening of individuals to determine and confirm exposure to various toxins and/or agents. Thus, the present invention is not meant to exclude any application outside of the medical field. Furthermore, the present invention is well suited to test any subject including, but not limited to, employees, workers, athletes, EMS personnel, emergency first responders, and any other subject who can be exposed to various agents or is required to be screened for exposure to any agent. For example, the present invention can be used by lawn care workers or crop dusters that can have been exposed to organophosphate pesticides or other toxic chemicals. Additionally, the present invention can be utilized to monitor pesticide exposure in the civilian population due to insufficiently washed fruits and/or vegetables.

The present invention can be used to detect any desired agent. For example, the device of the present invention can be used to detect agents in order to diagnose diseases or detect the presence of toxins or pollutants. The following list is meant to include, but is not limited to methods to detect, biological contaminants, chemical contaminants, environmental pollutants and toxins, radiation, effects of chemotherapy, levels of bilirubin, drug effectiveness, disease states, the amount of an allergic reaction, and to specifically determine the toxin.

For example, the present invention can be use to determine the presence of disease. Examples of such diseases include neurodegenerative diseases or other conditions are selected from the group consisting of adult-onset dementias such as Alzheimer's disease, Parkinson's disease, Huntington's Disease, Amyotrophic lateral sclerosis or motor-neural degenerative diseases like Myasthenia Gravis that are related to AChE expression. Additionally, AChE can be used to determine whether an individual has been exposed to a chemical or biological toxin or contaminant. Modulation in the AChE levels is indicative of such an exposure. Alternatively, the present invention can be used to detect the presence of cancer. In other words, the device can be used to detect the presence of markers of cancer.

The device of the present invention functions in the manner described above. The device functions by including, within the chamber, an analyzer capable of detecting the desired agent. The analyzer can be an assay that provides a detectable response in the presence of the desired agent. For example, the assay can include a labeled substrate bound to the chamber such that upon the addition of the agent the label becomes detectable. Examples of such assays are known to those of skill in the art and can include, but are not limited to, a competitive assay, an ELISA-type assay, or other similar assays. Alternatively, the analyzer can be a chemical or other reaction that occurs when the agent is present in the chamber.

The device of the present invention can also be used to monitor contaminants in a non-human sample. For example, the device can be used to monitor contaminants in water samples. In this embodiment, the device is adapted to either be located within or adjacent a water sample. In other words, the device can be placed in a location wherein water passes through or adjacent the device, e.g. the device can be modeled to affix to a faucet and is not attached using the transdermal patch disclosed above. The device includes a label that indicates the status of the water. Thus, if the water is contaminated, the label indicates such a changes. In the preferred embodiment, the label is a colorimetric indicator such that the color of the device changes corresponding to changes in the water. The ability to easily and inexpensively determine whether a water source is potable can prevent numerous illnesses and deaths that result from individuals inadvertently drinking contaminated water, which often occurs subsequent to disasters such as tornadoes or when water mains are broken. In these instances, it is often hours later that it is determined that the water flowing to individual's homes is not potable. The present invention eliminates this problem.

Further, a similar device can be used in conjunction with foodstuff. The device of the present invention can also be used to determine whether or not food has been contaminated. The device can detect whether a toxin has been introduced to the food or if the food has spoiled. The ability to determine this without extensive testing is beneficial for the safety of the general population and can prevent numerous illnesses and deaths resulting from individuals inadvertently eating contaminated food. For example, if meat is recalled as a result of contamination, then the device of the present invention provides individuals with their own indicators for meat quality. Alternatively, the device can be sold in units for use in the home. Examples of such uses include, but are not limited to, inclusion during canning, inclusion when storing leftovers after a meal, and as a tester for foods previously purchased that do not include the device.

While specific embodiments are disclosed herein, they are not exhaustive and can include other suitable designs and systems that vary in designs, methodologies, and transduction systems (i.e., assays) known to those of skill in the art. In other words, the examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

Production of One Embodiment of the Device of the Present Invention

FIG. 2 shows the CAD layout of the chambers wherein two chips constitute the top and bottom of the device. The bottom (FIG. 2a) chip measures 18 mm square after separation from the rest of the wafer. The circular chambers and diagonal capillaries are 200 μm in depth. The vertical lines in (FIG. 2b) of the device are air escape capillaries measuring 1 μm in depth and 2 μm in width. The two parts of the sensor unit are bonded face to face, producing the micro-fluidic device from the two micro-fluidic glass chips. The CBMD device was designed using Tanner Research, Inc. CAD tools, and is produced in Borofloat glass using MEMS based micro-machining techniques.

The micro-fluidic device utilized different diameter conduits to provide fluid flow into the sensing chambers, and allow air to escape while maintaining liquid in the assay chamber. To provide this information and data, a model of the conduits can be created, which consisted of cylindrical chambers and channels, and can be analyzed using equations for surface tension. The maximum pressure in a liquid droplet (ΔPᵢ (max)) at the center of the device is given by the surface tension (σ) and the radius of the hole (Rₕ), as shown in the following equation.

$$Δ {P}_{i} ( max ) = \frac{2 σ}{{R}_{h}}$$

Determining the pressure at the leading edge of the fluid in the channels of depth (d) and width (w) also requires the surface tension (σ) and the contact angle (θ).

$$Δ {P}_{a} = 2 σ cos ( θ ) ( \frac{1}{w} + \frac{1}{d} )$$

Using a surface tension of 72 dyne/cm for water and a hole radius of 250 μm, the pressure difference calculated at the liquid droplet at the center is 0.057 mPa. The contact angle was assumed to be 30 degrees, which correlates to untreated glass. The pressure necessary to force a liquid through a 10 by 10 μm channel is 2.5 mPa. Although, this is a significantly higher pressure than is necessary to flow through a 250 μm conduit, it can be necessary to isolate a hydrophobic surface treatment in the small air vent channels to retain the liquid in the sensing chambers.

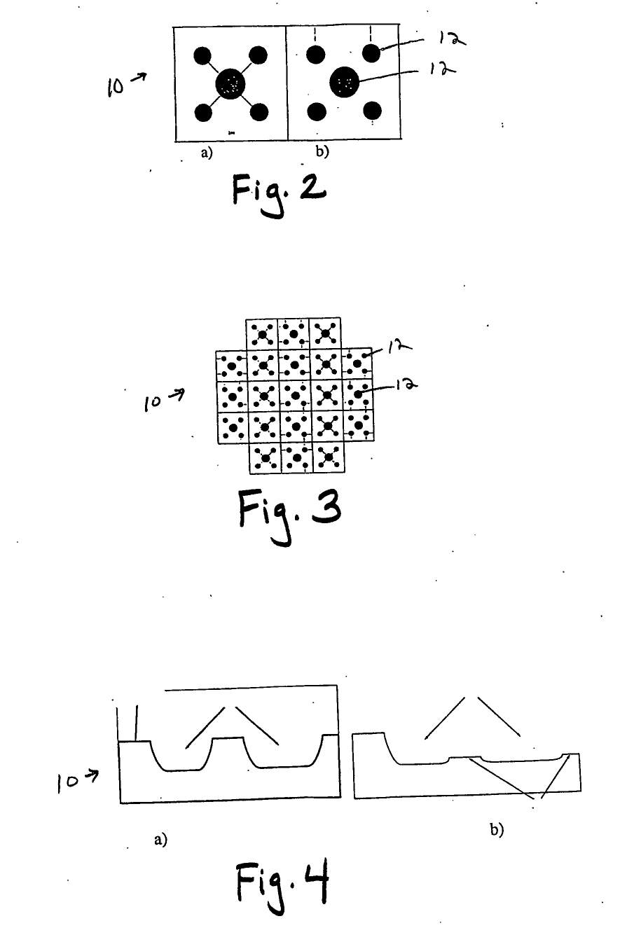

Internal design reviews of the CAD layouts were performed to ensure conformation to the manufacture requirements by Photronics, Inc.'s mask production process, as well as to insure appropriate and efficient utilization of all space available by incorporating a variety of test channel widths and depths. Electrical and chemical engineers examined the mask to ensure the designs were optimized, that no items were missing, and that the structures could be utilized to validate and verify the chemical engineering and fluid flow models. The complete mask layout is shown in FIG. 3.

These masks were utilized to photolithographically define the micro-fluidic conduits and air holes. The patterns were translated to the glass utilizing a masking and etching process.

The MEMS micro-fluidic processing of the glass wafers requires a two-mask step process. The first mask is utilized to generate the micro-fluidic chambers and conduits in the manner described above. Briefly, gold is deposited on both sides of the Borofloat glass wafers. The gold-coated glass wafer is patterned on one side in the conformations of the CAD generated mask design. The thin layers of gold are etched, which then serves as a mask for the glass chambers and conduits. The glass substrate is etched 200 μm deep utilizing a solution of 7:3 HF:HNO₃. Following the etching of the glass, the gold mask layers are removed.

The second mask step is utilized to produce the air escape holes. These are generated using a second mask layer to photo-pattern the 2 μm wide conduits using a standard photoresist. These air escape conduits are then etched to the desired depth using a buffered HF wet etch to etch the glass 1 μm to 20 μm deep into the surface from the sensor chamber to the scribe lane (approximately 2 mm). Because of the extremely small diameter of these holes, air can escape, but fluid is not able to pass through due to the high pressure required to flow through such small outlets.

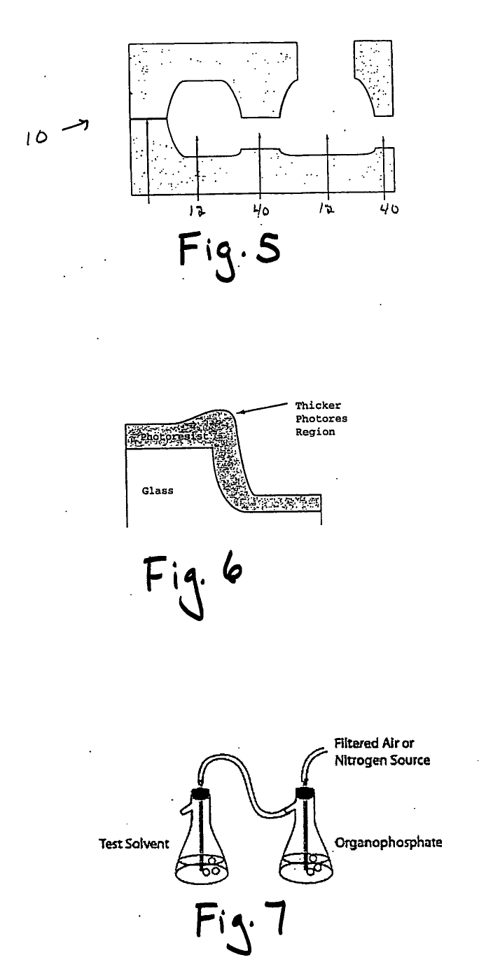

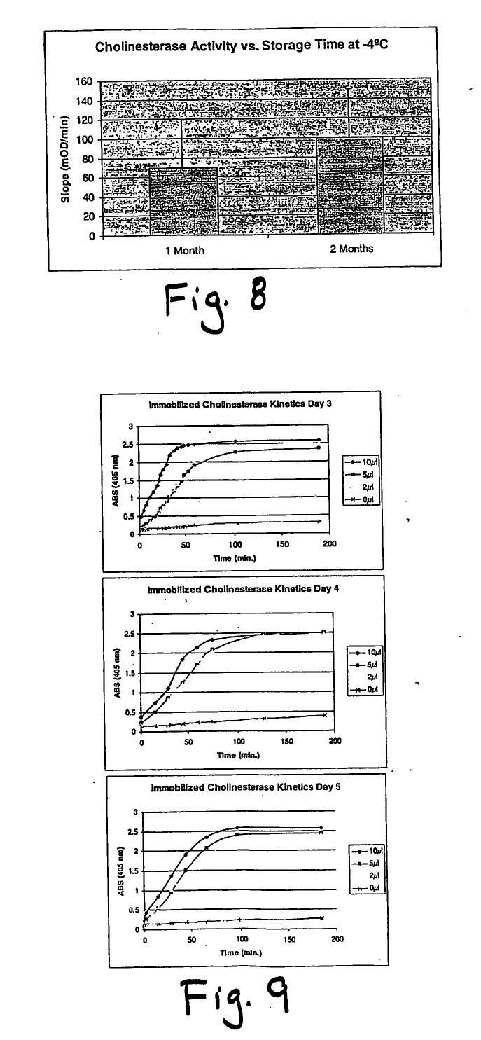

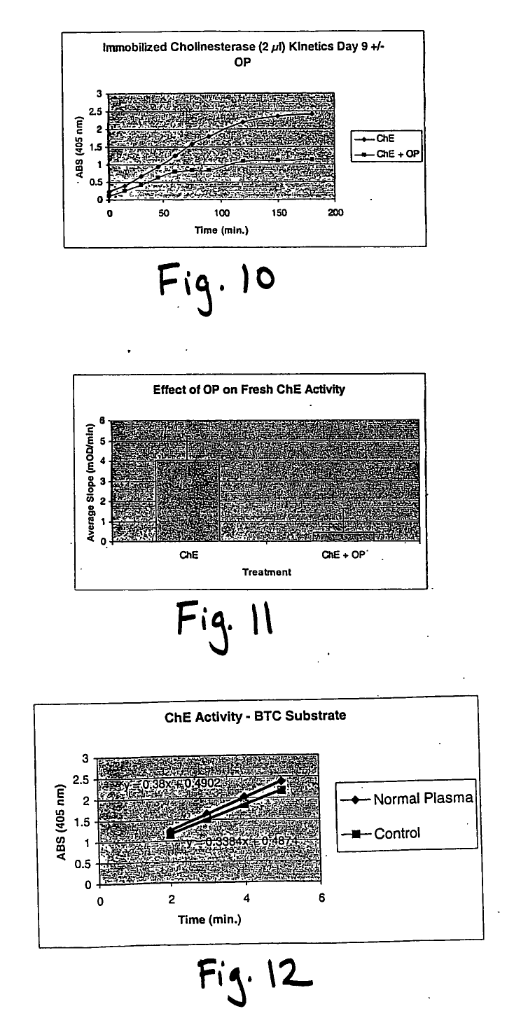

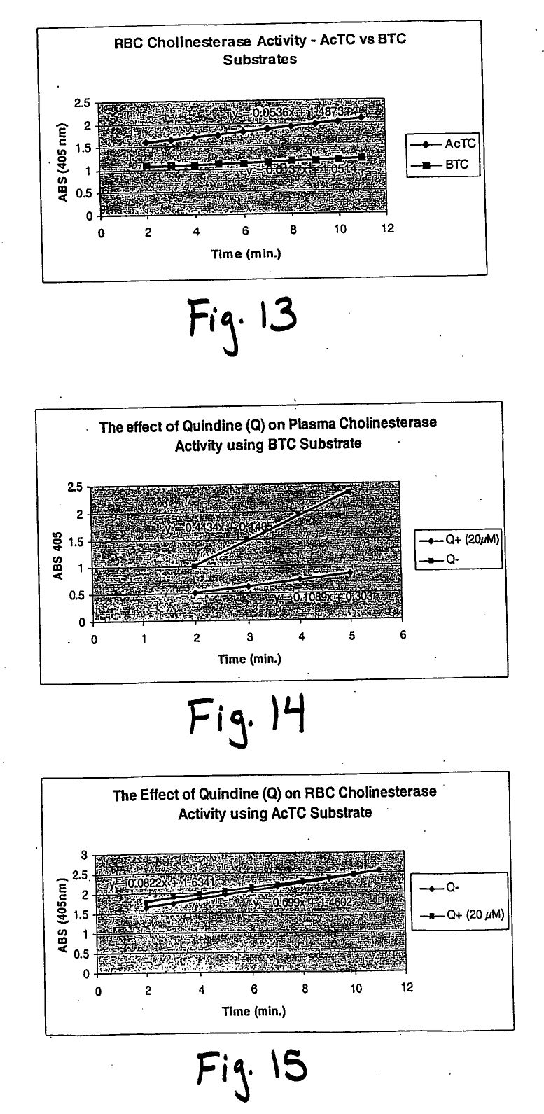

After the MEMS based micro-fluidic glass wafer processing, the chips are sawed into separate units, and the two parts of the device are assembled. The cross-section of the assembly process is shown in FIGS. 4 and 5. FIG. 5 shows top and bottom pieces of the chamber, mated together. Holes are then drilled into the sampling port. The sample under test is drawn from the sampling chamber into the sensor chambers utilizing capillary action.

Using micro-fluidic chemical engineering analysis, it has been calculated that the capillary action forces are sufficient to flow solution through the conduits utilizing no external pressure. Further, it was determined that these capillary forces are sufficiently high to force the solution through the large solution conduits, but is not great enough to force the liquid through the air escape holes with a more hydrophobic surface. To empirically test the chemical engineering and fluid flow models, several different air escape hole diameters were designed (2 μm, 5 μm, 10 μm, and 20 μm). To empirically test the chemical engineering and fluid flow models for the fluidic conduits, two different fluidic die were created and placed on the mask; one with a conduit diameter of 100 μm and the other with 200 μm. This initial prototype allowed 20-30 μl of sample to be dispensed into the center, and through capillary action, the 4 chambers were filled to initiate the assays, in duplicate, with positive and negative controls.

There is a difficulty found in utilizing this technique. The problem involved a thick bead of photoresist material at the corner of the etched features as pictured in FIG. 6. When exposing this region for photopatterning, it was necessary to increase the exposure time in order to expose the photoresist in this region all of the way through. However, this over exposure produced rough edges in the two-micron air hole conduits, since the photoresist was significantly overexposed, and developed diffraction patterns.

To overcome this difficulty, the masking order is reversed and excellent results are obtained (it was determined that it was possible to produce the devices with the proper features utilizing a reversed mask order). First, the shallow air escape holes were etched at a variety of depths from 2 μm to 20 μm, followed by the deep micro-fluidic conduit production, which includes the Cr/Au mask deposition, masking, and the deep etch.

### Example 2

Cholinesterase Activity Characterization

The device is based on the miniaturization and adaptation of the cholinesterase chemistry described below. To reduce the size and increase the ease of use, the reactants were dried and/or immobilized at the MOPAD sensing sites. Applicants have examined methods to optimize the immobilization procedure of the substrates, PTC/BTC and DNTB, at the sensing site including: lyophilization (freeze-drying), air-drying, and immobilization of the enzyme using a 2.5% glutaraldehyde solution. The immobilization process yielded promising data. The enzyme was added in excess and immobilized on a microtiter plate. After substrate was added, washed, and added again, the enzyme activity remained nearly constant. This has been repeated several times with three washes between each substrate addition. Such a system, when integrated into the MOPAD, enables continuous monitoring over long periods of time, rather than being a single-use device. Chemical engineering modeling and analysis of the prototype device can be used to optimize adsorption and capillary flow of the organophosphate samples through the exposed membrane to the sensing site, as well as to calibrate the colorimetric responses. Additionally, shelf life and accelerated testing (i.e. high temperatures, high humidity, etc.) can be examined utilizing the immobilized enzymes on the microtiter plate.

The organophosphate O,O-diethyl O-2-isopropyl-6-methylpyrimidin-4-yl phosphorothioate, (Diazinon, Sigma-Aldrich Cat. No. 45842) was purchased for initial testing. This compound is a common household pesticide often used for crawling insect extermination. The organophosphate was tested initially using a liquid sample. Using a 96 well microtiter plate, the Propyonyl-thiocholine substrate was added to the wells, followed by various dilutions of organophosphate, including a zero calibration point. The cholinesterase enzyme is then added to start the reaction and absorbance at 405 nm is measured at 30 second intervals. This experiment was repeated several times to derive a statistical mean and standard deviation. The data were analyzed by plotting the slope of the reaction versus OP concentration.

Air samples were also tested. The airborne samples were generated and collected utilizing a series of filtering flasks, which were connected together and used to bubble vaporized OPs through the appropriate solution. The first flask contains the organophosphate solution. Filtered air or nitrogen was bubbled through the OP, vaporizing it. The vaporized OP was turn bubbled through an appropriate buffer solution, causing the OP to dissolve back into the liquid to be assayed (See FIG. 7). The OP absorbed liquid was tested for cholinesterase inhibition in the same manner as described above for pure liquid samples.

A number of experiments were performed to characterize the kinetics of Cholinesterase (ChE) activity.
1. Determination of immobilized enzyme stability at −4° C.
2. Determination of enzyme/substrate kinetics and stability of the immobilized enzyme at room temperature
3. Determination of the effect of organophosphate on the enzyme kinetics of the immobilized enzyme
4. Determination of the effect of organophosphate on the enzyme kinetics of the fresh, non-immobilized enzyme
   Enzyme stability at −4 C

10 μl of 2.5% glutaraldehyde was added to 10 μl of the cholinesterase enzyme (ChE) preparation in 6 microtiter plate wells. This caused crosslinking of the enzyme and attachment to the plastic microtiter plate substrate. The wells were then tested, washed, and tested again, thereby providing a means to constantly monitor environmental preparations for OP activity with the same enzyme preparation.

The activity of the enzyme was determined by measuring the change in absorbance (or slope) after one month and two months of storage at −4 C (see FIG. 8). The slope of wells containing substrate alone was computed and found to be negligible.

After each slope determination, the plate was washed with two washes of distilled water and stored dry at −4 C. In some of the wells the immobilized enzyme mass lifted from the plastic well. Experiments were used to determine the effect of adding a cellulose acetate membrane to the well first, in an attempt to promote long-term adhesion of the enzyme. Separation of the enzyme globule from the plastic substrate caused the effective surface area of the immobilized enzyme to increase, enabling more substrate to react with the enzyme. This is the probable cause of the large variation in slope determinations as reflected by the large error bars in FIG. 9. Ultimately, this is a problem since the immobilized enzyme adheres better to the borosilicate glass of the MOPAD chamber.

Conclusion:

The immobilized enzyme preparation was extremely stable, having equivalent activity after two months of storage at −4 C.

Enzyme stability at room temperature

2 μl, 5 μi and 10 μl of enzyme were added to 12 microtiter wells, each with equivalent volumes of 2.5% glutaraldehyde to immobilize the enzyme. The plate was stored at −4 C for two days to dehydrate the immobilized enzyme and to improve the diffusion process. At various time intervals, 100 μl of substrate was added to each well and the plate was read, photometrically, at various time intervals for three hours (see FIG. 9). The plate was rinsed twice with distilled water and stored at room temperature for testing on the following day.

Conclusion:

At the enzyme/substrate ratios used, the enzyme activity remained for more than five days of storage at room temperature. The slope was linear for each of the readings and each of the volumes for the first 30 minutes of incubation. This preparation is stable for months at room temperature. Accelerated testing, utilizing high temperature and/or humidity storage, can be performed.

Effect of Organophosphate

The same plate that was used to test enzyme stability was used again to test the ability of organophosphate to inhibit the immobilized enzyme. Initially, to test the ability of a weak organophosphate commonly used by the general population, a commercial organophosphate insecticide was purchased and tested (Ortho Diazinon Ultra Insect Spray containing 22.4% Diazinon). This liquid preparation was diluted 1:10 in phosphate buffer. 10 μl of Diazinon was added to six of the twelve wells and 10 μl of phosphate buffer was added to the remaining six wells. 100 μl of substrate was added to each of the twelve wells to start the reaction. There was significant suppression of enzyme activity in the 2 μl immobilized enzyme wells (see FIG. 10).

Conclusion:

Significant inhibition of ChE activity was observed using 2 μl of enzyme, 10 μl of 2.24% Diazinon in phosphate buffer, and 100 μl of substrate. These same ratios of reagents were tested in the MOPAD device.

Effect of OP on Fresh, Soluble ChE

To confirm the ability of OP to inhibit ChE enzyme activity, a fresh, soluble enzyme preparation was tested. In an attempt to determine if varying the enzyme to OP ratio had an effect on inhibition, the enzyme and OP were diluted 1:100. This gave an enzyme to OP ratio 10 times less than in the first set of experiments. 2 μl of the diluted enzyme, 2 μl of the diluted OP and 100 μl of substrate were added to each of six microtiter plate wells. A kinetic protocol was created on the photometric microtiter plate reader to take an absorbance reading at 405 nm every minute for 10 minutes, and compute an average slope. The results of this experiment are shown in FIG. 11.

Conclusion:

Significant inhibition of ChE activity occurred using a fresh, non-immobilized preparation of ChE.

### Example 3

Artificial substrate Butyrylthiocholine (BTC) or acetylthiocholine (ACT), depending on whether BuChE or AChE is to be detected, is hydrolyzed in presence of the active enzyme to form thiocholine, which reacts with 5,5′-dithiobis-2-nitrobensoic acid (DNTB) to form yellow 5-thio-2-nitrobenzoate that possesses an absorbance peak at 405 nm. The rate of change in absorbance, A-405, is directly proportional to the cholinesterase activity.

The BTC, and the ACT enzymatic substrates and the controls were purchased from Sigma-Aldrich and used for the detection of plasma butyrylcholinesterase enzyme, also known as ‘pseudo’ cholinesterase, respectively for the ‘true’ acetylcholinesterase from RBCs. The enzymatic reactions were monitored using a BioTek Elx800 plate reader able to measure kinetic readings at 405 nm. The slope of the calorimetric reaction was computed and plotted as mOD units per minute.

Several tests were performed to evaluate the ability to photometrically detect cholinesterase activity in whole blood, lysed red blood cells, and plasma in a reaction volume of 3 μl. In addition, the ability to differentially detect acetylcholinesterase and butyrylcholinesterase activity from a single drop of blood was investigated. Note that in all graphs shown below, a least square linear regression was performed to determine the slope and intercept of the kinetic data and the equation appears next to the respective data. This was performed to provide an easy comparison of the difference in kinetics, if any. The equations follow the form: y=m×+b where m is the slope and b is the y intercept. Comparison of the kinetics between each treatment group can be made by directly comparing the slopes.

The first test was to verify the presence of cholinesterase activity in human plasma as compared to a commercially available plasma control.

Human whole blood was purchased from CBR Laboratories, Inc., Boston, Mass. The blood was centrifuged for 5 minutes at 7000 rpm (2500×g) and separated into plasma and red blood cells using a Fisher Scientific Centrific centrifuge (Model 225). The plasma was then transferred to 15 ml polystyrene tubes. The packed red blood cells were washed three times with saline buffer and lysed with a lysis buffer containing 155 mM NH₄Cl, 10 mM KHCO₃, 1 mM EDTA, and 170 mM Tris, at pH 7.3.

2 μl of plasma was transferred to a 96 well microtiter plate. 200 μl of butyrylthiocholine substrate was added to each well to start the reaction. Kinetic measurements were taken by measuring the absorbance at 405 nm every minute for five minutes. The average slope was computed. The results, depicted in FIG. 12, show almost identical slopes for control and plasma cholinesterase, confirming the capacity of the BTC substrate to detect cholinesterase activity in plasma.

The same test was also performed for lysed red blood cells to check the activity of the red blood cell cholinesterase, by using acetylthiocholine (AcTC) as enzymatic substrate.

As shown in FIG. 13, the acetylcholinesterase from RBC lysate had significant activity (slope=53.6 mOD/min) when the AcTC substrate was used, whereas there was significantly less activity (slope=13.7 mOD/min) for the reaction using the BTC substrate. The two enzymes hydrolyze both substrates but at different speeds. The minimal cholinesterase activity in the RBC lysate, using BTC substrate, is due to the cross-reactivity of the substrate.

These two experiments clearly demonstrate that AcTC substrate is specific for RBC cholinesterase and that BTC substrate is specific for plasma cholinesterase. When used in the BMCD system, these enzymatic tests allow for the differentiation of acute exposure to organophosphate toxins (increased plasma cholinesterase activity) and chronic exposure (increased RBC cholinesterase activity).

Quinidine (quinaglute), a class 1a antiarrythmic drug that is commonly used for atrial fibrillation cardioversion, also specifically inhibits plasma cholinesterase activity, but does not inhibit cholinesterase activity in red blood cells. For this reason, quinidine is useful for differentiating between plasma and RBC cholinesterase. To demonstrate this selective inhibition, plasma samples were treated with quinidine (20 μM) and the inhibitory effect was observed only when BTC was used (see FIG. 14).

The same experiment was performed using RBC lysate to determine the inhibitory effects of cholinesterase activity with and without quinidine. As expected, only a minute decrease in cholinesterase activity was observed (see FIG. 15).

The above experiments demonstrate that plasma and red blood cell cholinesterase activity can be measured in separated blood by measuring each fraction using BTC and AcTC substrates respectively. To demonstrate that the separation step is not needed in the final BCMD system, whole blood was lysed using the lysis buffer mentioned above and measured for butyrylcholinesterase and acetylcholinesterase activity. The results shown in FIG. 15 indicate that the two types of cholinesterase activity can be determined by using lysed whole blood. By using quinidine to inhibit butyrylcoholinesterase activity, red blood cell cholinesterase activity can be measured using AcTC substrate in one set of chambers. Accordingly, butyrylcholinesterase (plasma cholinesterase) activity can be measured using BTC substrate in the second set of chambers. The device can also incorporate a scheme of differential detection. The device can also be smaller and can incorporate its own optical detection system. At least three chambers can be used to detect each type of cholinesterase activity to give a statistically significant measurement.

The four components of AcTC substrate were prepared individually as follows: acetylthiocholine (3.8 mg/ml), DNTB (2.2 mg/ml), potassium phosphate (17.4 mg/ml) and triton-x (0.1%). 125 nl of each component was spotted onto the reaction chamber well and the solutions air-dried within 2 minutes. To start the reaction, 2.8 μl of plasma and plasma diluted 1:10 with 0.9% sodium chloride, were added to each of the four reaction chambers. The absorbance was read every 15 seconds for 2.5 minutes. FIG. 16 shows the results of this experiment. The data shows that diluted and undiluted plasma showed cholinesterase activity using substrate reagents that were dried and spotted individually. Using dried reagents in the BCMD device decreases the weight of the device while increasing its shelf life. Freeze-drying (lyophilization) can also be used to increase the stability of the reagents.

In summary, based on the results shown above, it is possible to measure both plasma and red blood cell cholinesterase activity in a single drop of blood. If unforeseen problems appear during the differential detection using this technique, other separation methods can be considered. MEMS techniques can be developed for the separation, providing a set of filters for the separation. Another possible solution is to implement the Pall Corporation's (East Hills, N.Y.) vertical separator of blood. Based on a chromatographic procedure, a blood drop is placed on a special media and, in 10 seconds, the separation occurs with the pure plasma eluting first, followed by the different cellular components.

In conclusion, these experiments have proven that the cholinesterase enzyme chemistry can be miniaturized using 3 μl volumes of whole blood samples. Dried substrate reagents at 125 nl volumes can be used without affecting the efficiency of the reaction. Coupling the miniaturized enzyme chemistry with AST's photodiode detection system and MEMS based micro-fluidic pumping system results in a miniaturized, lightweight, wearable unit for detecting blood cholinesterase.

Visual Calorimetric Change Analysis of Cholinesterase Activity in Samples Using Commercial Standards.

To demonstrate feasibility, the device was designed to visually detect colorimetric changes in the Ellman chemistry adapted for small volumes. Although the change in color, using this small volume, is visible to the naked eye, it was not possible to quantitate the change. For this reason, the detection chamber was designed and adapted to fit into a conventional 96 well plate and read using a conventional spectrophotometer (see FIG. 17).

The device can incorporate its, own optical detection system positioned exactly under each of the detection chambers.

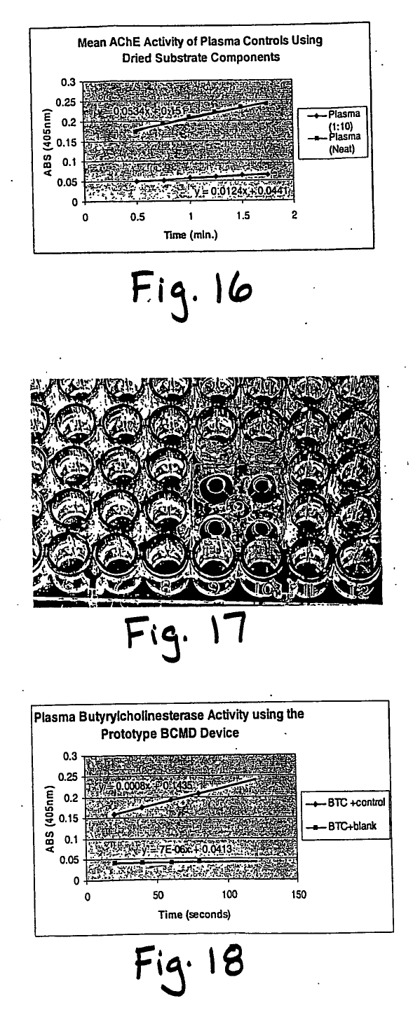

An experiment was performed to test the ability of the prototype device to detect calorimetric changes due to cholinesterase activity. In this experiment, 300 nl of plasma was added to two wells of the device and two wells contained no enzyme. 3 μl of BTC substrate was added to each of the four wells to start the reaction and the absorbance was recorded for every fifteen seconds. The absorbance increased in a linear manner for the wells containing plasma and clearly shows that a detectable color change occurred (see FIG. 18).

### Example 4

Analyze Interstitial Fluid Samples for Melatonin

Melatonin EIA:

The fluid samples were assayed using a commercially available direct saliva melatonin EIA (American Laboratory Products, Cat. No. 001-EK-DSM). This is a competitive binding assay. The samples, controls, and standards are incubated with melatonin biotin conjugate for three hours and a binding competition for a melatonin antibody, which is bound to the microtiter plate, occurs between the melatonin conjugate and the melatonin in the samples. The more melatonin that is present in the sample, the less biotin conjugate is bound. After three hours the plate was washed and enzyme label was added for one hour during which time binding between the conjugate and enzyme occurs. After one hour, the plate was washed and TMB substrate was added. The substrate is converted to a chromophore that absorbs light at 450 nm, in proportion to the amount of enzyme present. The more that light is absorbed indicates that less melatonin was present in the sample. Stop solution is added after a thirty minute incubation, and the plate was read using a BioTek EL800 microplate reader.

Results:

Melatonin

The concentrations of melatonin in the samples and controls were computed using the 4-parameter logistic model available in the BioTek KC Junior software. To normalize the data, the concentrations from the pre-melatonin saliva and interstitial fluid samples were subtracted from those obtained after melatonin ingestion. This gave melatonin values that were due solely to melatonin ingestion and removed any background readings due to cross reactivity to other interstitial fluid or saliva components as well as any background measurements due to the matrix of the iontophoresis electrode buffer itself.

Four out of the five volunteers showed an increase in interstitial fluid melatonin after ingestion (mean=9.0+/−6.2 pg/ml). Five out of the five volunteers showed an increase in saliva melatonin ranging from 110.8 to >324 pg/ml. The results are listed in Table 4.

TABLE 4
Comparison of saliva and interstitial fluid (I.S.F.) melatonin
concentration from the clinical trial samples.

| | Saliva Melatonin | |
|---|---|---|
| Volunteer | (pg/ml) | I.S.F. Melatonin (pg/ml) |
| 1 | 251.456 | 9.736 |
| 2 | >384 | 1.512 |
| 3 | >384 | — |
| 4 | 174.412 | 8.036 |
| 5 | 101.304 | 16.632 |

To assess and confirm the reliability of the sampling and immunoassay analysis and to correlate to literature values, the pre melatonin saliva values were averaged (n=5, mean=17.5+/−8.4 pg/ml). This compares to approximately 8 pg/ml of melatonin that is normally observed in saliva samples at 8:00 PM, the time that the pre melatonin samples were collected (See FIG. 19).

The volunteers were composed of males and females, age range 20 to 52 years old. The difference between interstitial fluid and saliva concentrations is due to the dilution of the melatonin that occurred when 2.0 ml of NaCl solution was added to the interstitial fluid collection electrode patch. The samples were diluted with 2.0 ml of NaCl giving a surface are to volume ratio of 7.0 cm²/2.0 ml=3.5 cm²/ml.

Glucose Monitoring

As an approximation of the dilution factor introduced by using a rather large volume (2.0 ml) of 0.9% NaCl in the iontophoresis electrode, the saliva and interstitial fluid samples for two of the volunteers, collected at 8:00 P.M. were assayed for B-D-glucose (see Table 5). The Amplex Red Glucose Assay Kit (Molecular Probes, Cat. No. A-12210) was used for this determination.

TABLE 5
Glucose concentrations for saliva and
interstitial fluid (I.S.F.) samples.

| Volunteer | Saliva Glucose (μM) | I.S.F. Glucose (μM) |
|---|---|---|
| 4 | 2.262 | 2.295 |
| 5 | 7.351 | 1.829 |

The results for the saliva glucose concentrations correlate with literature values of <20 μM as the basal glucose saliva concentration in normal human subjectsⁱ. Since fasting glucose levels normally observed in interstitial fluid samples are in the 5 mM rangeⁱⁱ, the collection of the interstitial fluid samples using 2.0 ml of 0.9% NaCl for the given electrode area, iontophoresis time, and dosage resulted in a concentration of glucose 1000 times lower than normally observed. This dilution explains the large difference between the saliva and interstitial fluid melatonin concentrations after melatonin ingestion.

Summary of Results

There were increases in interstitial fluid melatonin concentration after ingesting a melatonin tablet, however, they were not as dramatic as those seen in the saliva samples. The dilution factor introduced by adding 0.9% NaCl to the iontophoresis electrode collection pads was estimated by measuring glucose in the same samples. It was concluded that the interstitial fluid samples were diluted approximately 1000 times. This dilution problem can be overcome by using a device that has a surface to volume ratio 150 times greater than the commercial device used. In normal adults, serum melatonin concentrations are highest during the night (about 60 to 200 pg/mL) and lowest during the day (about 10 to 20 pg/mL). These concentrations are well within the melatonin standard curve as determined by amperometry (see FIG. 20).

Amperometric Measurement of Melatonin:

A melatonin (Sigma, Cat. No. M-5250, F.W. 232.3) standard curve was prepared with concentrations ranging from 25 pg/ml to 600 pg/ml. Cyclic voltammetry was performed using +/−900 mv, 200 mHz cycles, with oxidatively derived current flow captured at 300 mv versus a silver/silver chloride reference electrode. Cyclic voltammetry provided a linear function for the entire range of concentrations, proving that it is an appropriate technique for monitoring melatonin. There are several advantages to using cyclic voltammetry to assay melatonin. These include the rapidity of detection and quantification (seconds), the sensitivity for melatonin, the limit of detection for melatonin, and the ability to recycle the reaction (i.e. perform serially repeated assays for days).

Amperometric Measurement of Glucose:

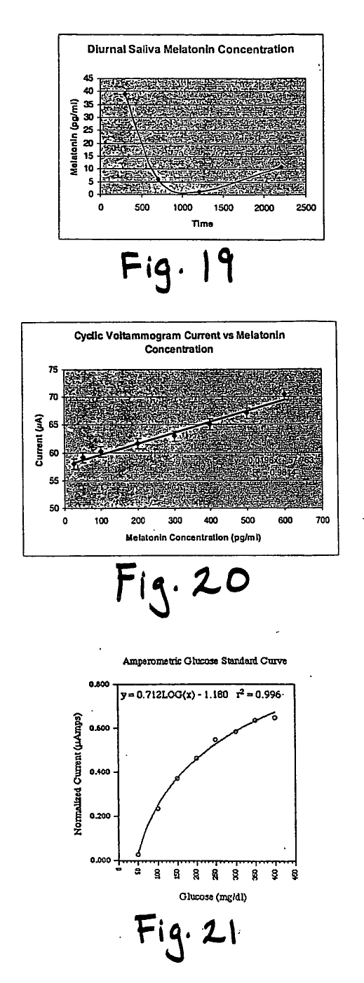

A glucose (Sigma, Cat. No. EC No 200-075-1, Lot No. 41K0184) standard curve (FIG. 21) was prepared with concentrations ranging from 50 mg/dL to 400 mg/dL. The glucose oxidase electrode was prepared by immobilizing glucose oxidase (Sigma, Cat. No. G-2133, Lot No. 110K1373) on a cellulose acetate (ACROS Cat. No. 17778-5000, Lot. No. B0057722) membrane covered electrode. Bovine serum albumin (BSA, ICN Cat. No. 840042, Lot. No. 2709E) was added to enhance crosslinking by 2.5% glutaraldehyde (ACROS, Cat. No. 111-30-8, Lot. No. A009956001). This gave a highly selective electrode to measure the production of hydrogen peroxide by glucose oxidase in the presence of glucose.

Cyclic voltammetry, incorporating AST's glucose oxidase electrode, was performed using +/−900 mv, 500 mHz cycles, with oxidatively derived current flow captured at 425 mv versus a silver/silver chloride reference electrode. Cyclic voltammetry provided a logarithmic function for the entire range of concentrations, proving that it is an appropriate technique for monitoring glucose. As with melatonin, there are several advantages to using cyclic voltammetry including the rapidity of detection and quantification (seconds), the sensitivity, the limit of detection and the ability to recycle the reaction (i.e. perform serially repeated assays for days).

### Example 5

Optical Sensors:

Optical chemical sensors require both a light source and detector in order to measure the color change of the enzyme containing membrane. The light source and detector required for single wavelength optical chemical sensing are implemented as a narrow band light emitting diode (LED) that can be incorporated into the MMCS device, and used to produce the wavelengths required for the optical measurement.

Several methods are available to produce optical detectors and photo-diodes. One of the most efficient is the utilization of Gallium Arsenide, however Gallium Arsenide wafers are extremely expensive and brittle, so they are only used in the most demanding optical applications. Standard CMOS processes can be used to produce these devices, however, in solution applications there is a tendency for the optically derived current to leak to the solution. Silicon-on-insulator (SOI) processes can be used to fabricate high quality photo-diodes, which are isolated from solution as well as other active circuitry for the formation of optical sensors on the same substrate. As an added benefit, SOI substrates are inherently radiation hard.

SOI photodiodes are produced by utilizing a lightly-doped, moderately-thin film (0.5 μm) SOI substrate, and masking the bulk implant as well as the well implant. Intrinsic regions remain wherever both implants are masked in the active areas, which can be used to produce p-i-n photo-diodes. When the sources and drains are implanted for the on chip circuitry, the n+ and p+ regions of the photo-diode are formed.

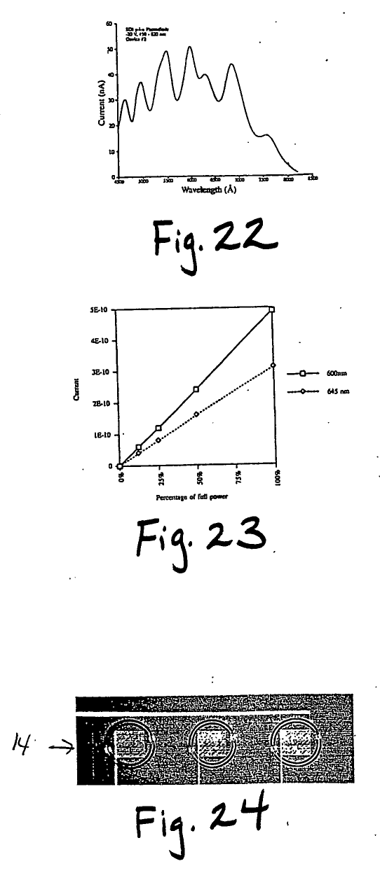

Since the photo-active intrinsic region is encapsulated on top and bottom by oxide, the diode acts as a quarter wave stack, enhancing the signal at certain wavelengths as shown in FIG. 22. The response of the diodes is linear to the amount of incident power, as shown in FIG. 23. The active area for the photo-diode responses shown in FIGS. 22 and 23 are 100 μm by 100 μm. To improve the sensitivity and associated power the active area of the photo-diodes can be increased. The response can easily be increased by a factor of 20 by increasing the area, since the minimum-sized membrane for an optical sensor is approximately 300 μm diameter; the diode can be as large as the membrane before it affects sensor size.

This fabrication process allows standard silicon processing steps to be used to produce electronic, sensor controlling, telemetric, and actuator controlling circuitry. The circuitry and the optical sensors are both intrinsically isolated from solution without consideration of external packaging.

The optical chemical sensors reproduced on silicon chips by incorporating a photo-diode with an optical membrane on top of the diode shown in FIG. 24. Using a narrow band light emitting diode (LED), color change can be determined. Various MEMS structures were developed to prevent the ion-selective membranes from flowing into neighboring membranes eliminating contamination, and improving reproducibility and yield. To reduce the size of the sensors and semiconductor chip, as well as reduce the noise, it was essential that integrated circuitry be combined with the chemical sensing devices thereby reducing noise from the high impedance signals associated with sensors. Customized processes were designed and developed that allow all sensor processing to be performed after standard CMOS processing, so that standard CMOS circuits could be utilized. Various micro-machining techniques were developed and utilized for the packaging of these sensors.

Amperometric Sensors:

Applicants developed a series of microscopic semiconductor sensor arrays with interdigitated amperometric and potentiometric sensors, funded by a NIH, NINDS, SBIR project (1 R43 NS37989-01). Sensor arrays with varying dimensions are on hand and can be used, free of royalty or licensing charges, for integration into the present invention.

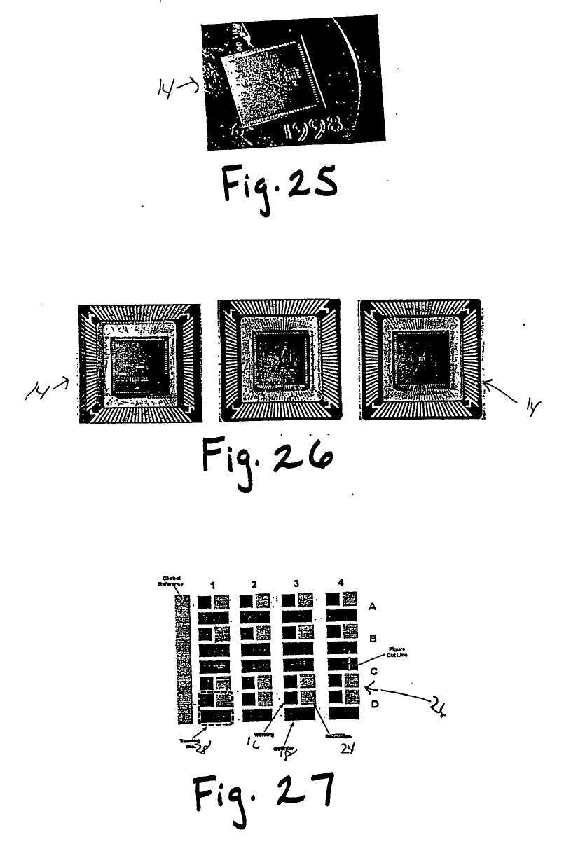

The sensors are capable of detecting a wide variety of bio-relevant molecules and ions. Through the use of asymmetric membrane chemistries, antibodies, and enzymes, these sensors can also detect a wide array of neutral molecules. Five conformations of sensor arrays, each incorporating 49 electrodes, were constructed using electrode sizes of 2, 4, 8, 32, and 100 μm. A photomicrograph of the 2 μm sensor array is presented in FIG. 25, different size sensor array chips bonded in a ceramic carrier are presented in FIG. 26, and a schematic of the sensor array is presented in FIG. 27.

The sensors in the array can be utilized in concert as: 1) ion selective electrodes (ISEs) capable of monitoring a wide variety of important ions including electrolytes, stress hormones, CO₂, local anesthetics, a variety of herbicides, heparin, medicinal drugs, lithium, etc. 2) amperometric electrodes employing chronoamperometry and cyclic voltammetry for the detection of more complex molecules, such as hormones, neurotransmitters, neurotoxins and other environmental contaminants, and 3) electrodes incorporating membranes with assay components that can be used to provide great sensitivity and selectivity, e.g. by immobilizing antibodies and/or enzymes on the surface of an ion-selective membrane and performing an enzyme-linked immunosorbent assay (ELISA) for example.

The schematic diagram in FIG. 27 illustrates that electrode orientation was altered from site to site. This permitted combination of electrodes from adjacent sites to act as a single larger electrode providing more flexibility to detect, verify, and analyze the effect of electrode size on the electrochemical response curves.

Additionally, AST has produced other sensor conformations, each with differing transduction and membrane encapsulation properties. These designs incorporate rectangular, circular, and concentric circle shaped electrodes (FIG. 28).

Cholinesterase Measurement:

Applicants successfully miniaturized a cholinesterase assay using immobilized ChE enzyme in volumes of 3 μl. Borofloat glass chambers were constructed using MEMS fabrication techniques. Chrome/gold masks were used to define the etch channels and the glass wafers were etched using standard photolithographic techniques.

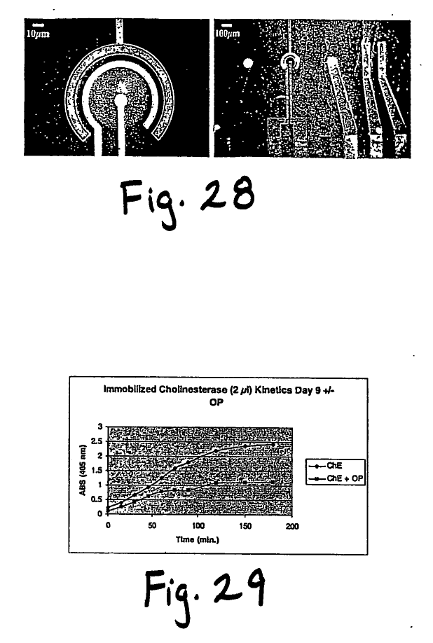

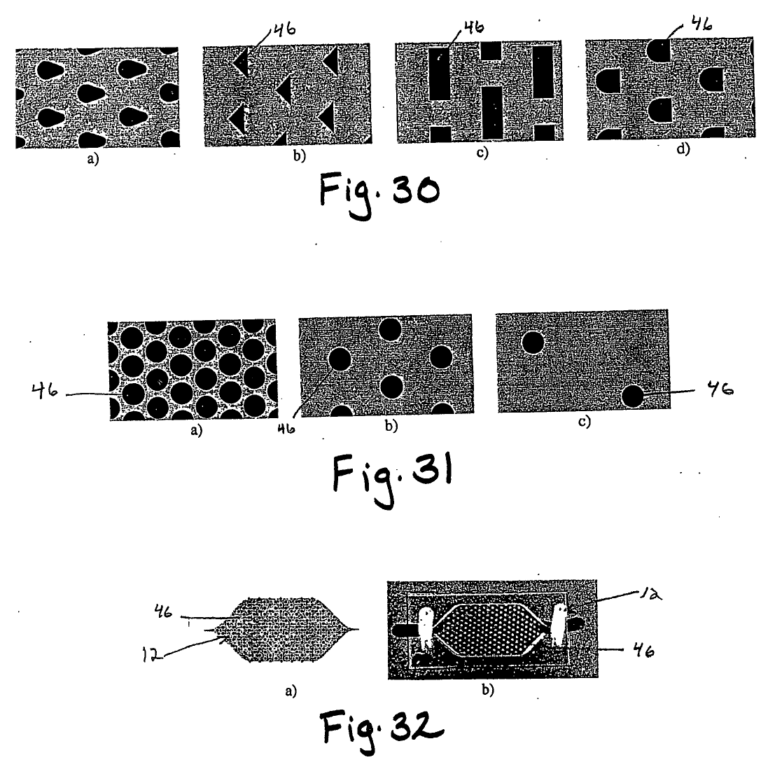

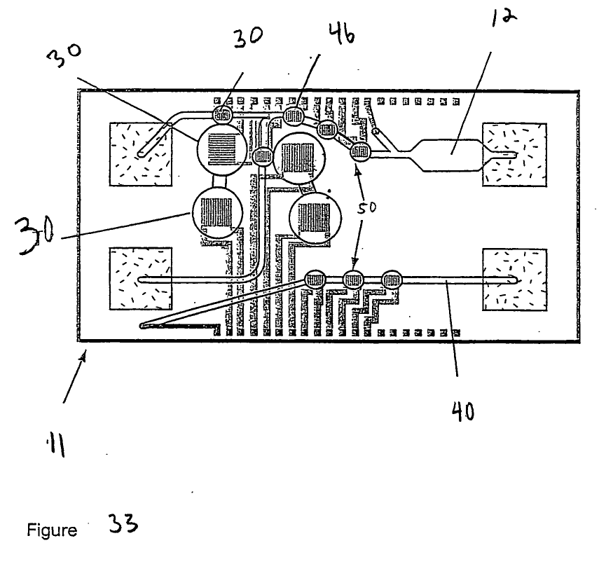

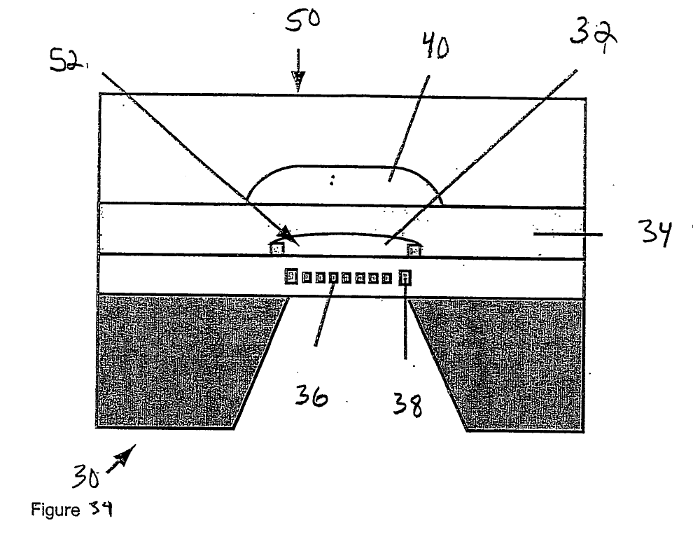

The chambers were used to successfully verify that a cholinesterase assay could be miniaturized. 3 μl volumes containing substrate were sufficient to record a change in light absorbance as the kinetic hydrolysis of the enzyme ensued. Moreover, an inhibition of the ChE activity was demonstrated in the presence of OP (see FIG. 29). To measure the change in optical density as the enzymatic reaction ensues, the chamber is mounted on a standard 96 well micro-titer plate and read using a BioTek Elx800 plate reader.

Testing of the Cholinesterase and OP Hydrolase Amperometric Sensors

Applicants have designed, constructed, and utilized a variety of microscopic amperometric sensor arrays for the detection of a wide variety of molecules. Briefly, cyclic voltammetry is a technique whereby a cyclically repeated triangular waveform of potential is applied between the working and counter electrode. Individual molecules have characteristic oxidation and reduction potentials based on their chemical moieties. When the voltage between the electrodes reaches the oxidation potential of a particular molecule, that molecule oxidizes. Oxidation is a process whereby an electron is stripped from the molecule. A third electrode, the counter electrode, absorbs the oxidatively produced electrons, effectively transducing chemistry into electricity. The flow of electrons per unit of time is current, which is proportional to the number of molecules being oxidized. The voltage at which this oxidatively produced current is obtained provides information useful for identifying the molecule being measured.

At solid stationary microelectrodes operating under conditions of cyclic voltammetry the peak current in microamperes, iₚ, is given for a reversible electrode reaction by the Randles-Sevcik equationⁱⁱⁱ:
iₚ=2.687×10⁵n^{3/2}AD^{1/2}Cv^{1/2}
where n=the number of electrons transferred
A=the electrode area in cm²
D=the diffusion coefficient of the electroactive species in cm² per second
C=the bulk concentration of the electroactive species in millimoles per liter
v=the scan rate of the applied cyclic voltage sweep in volts per second.

Cyclic voltammetry has several advantages over other amperometric techniques. During each cycle, the potential on the working electrode reverses and electrically cleans the electrode of molecules adsorbed during the previous cycle. The technique is quantitative for both oxidation and reduction.

Since the objective measure of concentration of the various G and V agents performed through the optical enzymatic reaction, cyclic voltammetry can be utilized to aid in the identification of the particular agent. Cyclic voltammetry has the capability of providing confirmation of the identity of an analyte by measuring its reduction potential as well as its oxidation potential. As the potential is scanned toward a negative potential, a cathodic peak is obtained due to reduction of the analyte, Ox, to form a reduced metabolite, Red, according to the following equation:
Ox+ne [symbol] Red
where ne is the number of electrons transferred in the reaction¹⁰. The voltage sweep then reverses direction and scans towards a positive potential. If the scan rate is sufficiently rapid, some of the Red produced by the cathodic sweep can still be in the vicinity of the electrodes and can be reoxidized to Ox, producing the anodic peak. For completely reversible reactions, the anodic and cathodic peak potentials are separated by the potential increment:
E_{anodic}−E_{cathodic}=0.059/n Volts
where n is the number of electrons involved in the oxidation and reduction.

Cyclic voltammetry provides the ability to measure the concentrations of several molecules sequentially in a single scan, as long as their oxidation potentials differ. For example, the concentrations of a wide variety of molecules can all be monitored sequentially from a mixture of these compounds; the value of oxidatively-derived current flow is captured at all potentials in the cyclic voltammogram.

In addition to oxidatively derived current measurement, the assessment of the class of analyte can be ascertained by providing selective molecular access to the electrodes by depositing membranes on them.

Various OP solutions can be used to determine the analytical performance and detection limits of the newly constructed OP sensors. The amperometric sensor results can be compared to those obtained by photometric detection.

Throughout this application, author and year and patents by number reference various publications, including United States patents. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used herein, is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

TABLE 1
Pyrex 50 micron depth

Volume of chamber without supports: 8.27 mm³
Volume range depending on number of supports: 5.8 mm³ to 8.27 mm³
Length of chamber 20 mm
Width of chamber 10 mm
Post height 50 microns
Circle post bottom diameter: .4 mm
Circle post top(skin side) diameter: .16 mm
Circle area against skin .0201 mm²
Circle post volume 0.0033 mm³
400 and 200 micron diameter ends of Tear drop
Tear drop bottom width 400 microns
Tear drop top (skin side) width 160 microns
Tear drop bottom length 600 microns
Tear drop top (skin side) length 360 microns
Tear drop area against skin .0361 mm²
Tear drop post volume 0.0072 mm³
Rectangle bottom length 1 mm
Rectangle top (skin side) length 0.76 mm
Rectangle bottom width 0.4 mm
Rectangle top (skin side) width 0.16 mm
Rectangle area against skin .1216 mm²
Rectangle post volume 0.013 mm³
Triangle bottom length 0.28 mm
Triangle top (skin side) length 0.01 mm
Triangle bottom width 0.56 mm
Triangle top (skin side) width 0.02 mm
Triangle area against skin .0001 mm²
Triangle post volume 0.002 mm³
Flat side circle bottom length 0.4 mm
Flat side circle top (skin side) length 0.16 mm
Flat side circle bottom width 0.4 mm
Flat side circle top (skin side) width 0.16 mm
Flat side circle area against skin .0229 mm²
Flat side circle post volume 0.0055 mm³

TABLE 2
Borofloat 50 micron depth

Volume of chamber: 8.27 mm³
Volume range depending on number of supports: 4.61 mm³ to 8.27 mm³
Length of chamber 20 mm
Width of chamber 10 mm
Post height 50 microns
Circle post bottom diameter: .4 mm
Circle post top (skin side)diameter: .3 mm
Circle area against skin .0707 mm²
Circle post volume 0.0048 mm³
400 and 200 micron diameter ends of Tear drop
Tear drop bottom width 400 microns
Tear drop top (skin side) width 300 microns
Tear drop bottom length 600 microns
Tear drop top (skin side) length 500 microns
Tear drop area against skin .1043 mm²
Tear drop post volume 0.0099 mm³
Rectangle bottom length 1 mm
Rectangle top (skin side) length 0.9 mm
Rectangle bottom width 0.4 mm
Rectangle top (skin side) width 0.3 mm
Rectangle area against skin .27 mm²
Rectangle post volume 0.0168 mm³
Triangle bottom length 0.28 mm
Triangle top (skin side) length 0.16 mm
Triangle bottom width 0.56 mm
Triangle top (skin side) width 0.32 mm
Triangle area against skin .0254 mm²
Triangle post volume 0.0025 mm³
Flat side circle bottom length 0.4 mm
Flat side circle top (skin side) length 0.3 mm
Flat side circle bottom width 0.4 mm
Flat side circle top (skin side) width 0.3 mm
Flat side circle area against skin .0803 mm²
Flat side post volume 0.0076 mm³

TABLE 3
Borofloat 100 micron depth

Volume of chamber: 16.54 mm³
Volume range depending on number of supports: 11 mm³ to 16.54 mm³
Length of chamber 20 mm
Width of chamber 10 mm
Post height 100 microns
Circle post bottom diameter: .4 mm
Circle post top (skin side)diameter: .2 mm
Circle area against skin .0314 mm²
Circle post volume 0.0073 mm³
400 and 200 micron diameter ends of Tear drop
Tear drop bottom width 400 microns
Tear drop top (skin side) width 200 microns
Tear drop bottom length 600 microns
Tear drop top (skin side) length 400 microns
Tear drop area against skin .0482 mm²
Tear drop post volume 0.0154 mm³
Rectangle bottom length 1 mm
Rectangle top (skin side) length 0.8 mm
Rectangle bottom width 0.4 mm
Rectangle top (skin side) width 0.2 mm
Rectangle area against skin .16 mm²
Rectangle post volume 0.028 mm³
Triangle bottom length 0.28 mm
Triangle top (skin side) length 0.0385 mm
Triangle bottom width 0.56 mm
Triangle top (skin side) width 0.077 mm
Triangle area against skin .0015 mm²
Triangle post volume 0.004 mm³
Flat side circle bottom length 0.4 mm
Flat side circle top (skin side) length 0.2 mm
Flat side circle bottom width 0.4 mm
Flat side circle top (skin side) width 0.2 mm
Flat side circle area against skin .0357 mm²
Flat side circle post volume 0.0124 mm³

TABLE 4
Borofloat 150 micron depth

Volume of chamber: 24.81 mm³
Volume range depending on number of supports: 18.58 mm³
to 24.81 mm³
Length of chamber 20 mm
Width of chamber 10 mm
Post height 150 microns
Circle post bottom diameter: .4 mm
Circle post top (skin side) diameter: .10 mm
Circle area against skin .0079 mm²
Circle post volume 0.0082 mm³
400 and 200 micron diameter ends of Tear drop
Tear drop bottom width 400 microns
Tear drop top (skin side) width 100 microns
Tear drop bottom length 600 microns
Tear drop top (skin side) length 300 microns
Tear drop area against skin .0202 mm²
Tear drop post volume 0.0196 mm³
Rectangle bottom length 1 mm
Rectangle top (skin side) length 0.70 mm
Rectangle bottom width 0.4 mm
Rectangle top (skin side) width 0.10 mm
Rectangle area against skin .07 mm²
Rectangle post volume 0.0353 mm³
Triangle bottom length 0.28 mm
Triangle top (skin side) length 0.01 mm
Triangle bottom width 0.56 mm
Triangle top (skin side) width 0.02 mm
Triangle area against skin .0001 mm²
Triangle post volume 0.0059 mm³
Flat side circle bottom length 0.4 mm
Flat side circle top (skin side) length 0.1 mm
Flat side circle bottom width 0.4 mm
Flat side circle top (skin side) width 0.1 mm
Flat side circle area against skin .0089 mm²
Flat side circle post volume 0.0154 mm³

1. A micro-device for testing agents in a minute amount of fluid, said micro-device comprising at least one micro-chamber; and testing means for testing for agents in the fluid, said testing means being located within said micro-chamber.

2. The device according to claim 1, wherein said micro-chamber includes collecting means for collecting the fluid and at least one reaction chamber in communication to said collecting means through at least one micro-conduit.

3. The device according to claim 2, wherein said micro-chamber is a single chamber.

4. The device according to claim 2, wherein said micro-chamber includes at least two micro-chambers.

5. The device according to claim 4, wherein said micro-chamber includes micro-conduits connecting said at least two micro-chambers.

6. The device according to claim 5, wherein said micro-conduits are selected from the group consisting essentially of micro-tubules and micro-fluidic capillaries.

7. The device according to claim 5, further including pumping means for pumping the fluid through said micro-conduits.

8. The device according to claim 1, wherein said micro chamber includes support means for supporting said micro-chamber.

9. The device according to claim 8, wherein said support means are configured in a shape selected from the group consisting essentially of tear drop, oval, square, rectangular, octagonal, and triangle.

10. The device according to claim 1, further including analyzing means for analyzing the fluid.

11. The device according to claim 10, wherein said analyzing means is selected from the group consisting essentially of a competitive assay, an immunoassay, an assay, a radioimmunoassay, an enzymatic assay, a potentiometry assay, an amperometric assay, an electrochemical reaction, and immunological reactions.

12. The device according to claim 1, further including a micro-fluidic system.

13. The device according to claim 1, wherein said device is hand-held.

14. The device according to claim 13, wherein said hand-held device includes digital display means for displaying the results of the testing.

15. The device according to claim 1, further including attachment means for attaching said device to a location in need of testing.

16. The device according to claim 15, wherein said attachment means is selected from the group consisting essentially of a patch, an adhesive, and a skin adhesive.

17. The device according to claim 1, wherein said device is a dipstick.

18. The device according to claim 1, wherein said device can be used to test to detect biological contaminants, chemical contaminants, environmental pollutants and toxins, radiation, effects of chemotherapy, levels of bilirubin, drug effectiveness, disease states, the amount of an allergic reaction, and to specifically determine the toxin present in the fluid.

19. The device according to claim 18, wherein said device is used to detect an agent in vivo.

20. The device according to claim 18, wherein said device is used to detect an agent in vitro.

21. A micro-electro-mechanical system for testing for agents in a fluid, said system comprising at least one micro-chamber; and a microfluidic system for acquisition of fluid into and throughout said micro-chamber.

22. A micro-device for testing for agents in a fluid, said device comprising a miniature sampling chamber for testing for agents in a small amount of fluid.
